(19) United States
(12) Patent Application Publication
(10) Pub. No.: US 2012/0315329 A1
(43) Pub. Date: Dec. 13, 2012

(54) SILOXANE SURFACE-MODIFIED HYDROGEL AND HYDROGEL MICROPARTICLE COMPOSITIONS

(75) Inventors: Dongchan Ahn, Midland, MI (US); James Thompson, Sanford, MI (US)

(73) Assignee: Dow Corning Corporation, Midland, MI (US)

(21) Appl. No.: 13/579,017

(22) PCT Filed: Feb. 17, 2011
(86) PCT No.: PCT/US11/25249

§ 371 Date: Aug. 14, 2012

Related U.S. Application Data
(60) Provisional application No. 61/305,859, filed on Feb. 18, 2010.

(51) Int. Cl. A61K 47/34; A61K 9/48; A61K 31/455

(52) U.S. Cl. 424/463; 514/772.1; 514/355

(57) ABSTRACT

Embodiments of the present invention relate generally to siloxane surface-modified hydrogel microparticles and pastes, methods for their preparation, and uses thereof for delivery of personal care and healthcare active ingredients, as well as agricultural active ingredients.

### BACKGROUND

Hydrogels can be generally characterized as having a cross-linked polymer matrix (elastomer) component that swells when contacted with water or a sufficiently water compatible fluid Polymeric microparticles that have become swollen due to the fluid trapped within the matrix are referred to as hydrogel microparticles. Since active ingredients and other compounds can be contained within the matrix, hydrogels and hydrogel microparticles (as well as pastes and powders made therefrom) have been found to be useful for the encapsulation and delivery of such compounds in a variety of applications. For example, hydrogels and hydrogel microparticles are particularly useful for encapsulation and delivery of pharmaceutical agents, vitamins, fragrances, oils, and other compounds in personal care and healthcare applications. In particular, hydrogels and hydrogel microparticles are useful for absorption and delivery of water-soluble and alcohol-soluble actives. However, such hydrogels and microparticles may have limited resistance to pre-mature release of the actives contained therein when exposed to aqueous and/or alcohol systems. Pastes made from hydrogel compositions are typically stable and can have a wide range of viscosities, thereby making them particularly useful as bases for certain applications.

Although various methods of preparing hydrogels, and hydrogel microparticles are known, there remains a need for methods of readily modifying hydrogels and hydrogel microparticles for use in a variety of applications. For example, there remains a need for methods of adapting a water-dispersible hydrogel or hydrogel microparticle to modulate the ingress or release of water from or to its environment.

### SUMMARY

These needs are met by the present invention, which in various embodiments provides siloxane surface-modified hydrogels and hydrogel microparticles, methods for their preparation, and uses thereof.

In various embodiments, provided are methods for the preparation of siloxane surface-modified hydrogels and hydrogel microparticles, comprising: treating a hydrogel or hydrogel microparticles with Component (A), at least one amino-functional organosilicon compound, to form at least one siloxane-coated surface on the hydrogel or hydrogel microparticles; wherein the hydrogel or hydrogel microparticles comprise Component (B), at least one organic polymer comprising amine-reactive groups selected from carboxy-functional groups, sulfonic acid-functional groups, epoxy groups, or combinations thereof; and wherein the hydrogel comprises Component (C), at least one absorbable solvent selected from water, water-compatible alcohols, and combinations thereof; or the hydrogel microparticles are treated in the presence of Component (C).

In various embodiments, provided are pastes comprising siloxane surface-modified hydrogels prepared by the provided methods. In various embodiments, also provided are siloxane surface-modified hydrogel microparticles prepared by the provided methods.

These and additional features and advantages of the invention will become apparent in the course of the following detailed description.

### DETAILED DESCRIPTION

Features and advantages of the invention will now be described with occasional reference to specific embodiments. However, the invention may be embodied in different forms and should not be construed as limited to the embodiments set forth herein. Rather, these embodiments are provided so that this disclosure will be thorough and complete and will fully convey the scope of the invention to those skilled in the art.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

Unless otherwise specifically indicated, the term “water-compatible” as used in the specification and appended claims is intended to mean at least partially soluble in water, but when used to describe a cross-linked polymer, the term is intended to mean that the polymer is able to absorb water.

As used in the specification and appended claims, the term “hydrogel” is intended to refer to gels in which the cross-linked polymer matrix is fully or partially swollen with water, one or more water-compatible alcohols, or combinations thereof. Accordingly, the term also includes, but is not limited to, alcogels fully or partially swollen with a water-compatible alcohol. The crosslinking of the polymer matrix may be chemical or physical in nature. As non-limiting examples, the hydrogel may be crosslinked through covalent bonds, ionic interactions, hydrogen bonding, chain entanglement, or self-association of microphase segregating moieties. Additionally, it is to be understood that such hydrogels may exist and be used in a dehydrated (unswollen) state.

Unless otherwise specifically indicated, the term “hydrogel microparticle” is used in the specification and appended claims is intended to refer to both a polymeric microparticle and a polymeric microparticle that is swollen with a sufficiently compatible fluid.

As used in the specification and appended claims, the term “alcohol” is intended to refer to water-compatible alcohols. Accordingly, the term “alcohol-compatible organic polymer” is intended to refer to an organic polymer that is compatible with a water-compatible alcohol.

As used in the specification and appended claims, the term “hydrophobic” is intended to mean lacking an affinity for and/or being resistant to water and/or water-compatible compounds. Accordingly, the term also refers to lacking an affinity for and/or being resistant to water-compatible alcohols.

The term “paste,” as used in the specification and appended claims, is intended to mean a suspension of hydrogel microparticles in a fluid.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

Water soluble polymers and hydrogels are useful for thickening, or gelling, water and/or alcohols, as well as materials that are compatible with water and/or alcohols. Hydrogels are used as carriers or dispersants for a variety of applications in which water or alcohol-soluble active ingredient are introduced to the surroundings. For controlled release applications where it is desirable to control the rate of delivery of the active ingredient to the surroundings, hydrogels prepared by known methods suffer from the limitation of not providing significant control of active diffusion rates. Further, many of the most common hydrogel materials, such as polyacrylic acid derivatives, tend to be difficult to handle both as dehydrated neat solids (due to fine particle size and extreme moisture sensitivity) and as partially or fully hydrated gels because of their tackiness. Hence, it is considered beneficial to provide a hydrophobic barrier to slow the diffusion of the active ingredients out of the hydrogel matrix into the surroundings and to improve handling characteristics.

In various embodiments, provided are hydrogels and hydrogel microparticles with siloxane surface coatings that act as barriers for the migration of water and water-compatible compounds, methods for their preparation, and uses thereof. In various embodiments, provided are methods of forming siloxane coatings on the exposed surfaces or near-surface regions of hydrogels and hydrogel microparticles, wherein the coating selected allows for modulation of rates of transport of water and water-compatible components across the coating. In some embodiments, when the method is carried out on a monolithic hydrogel, an siloxane coating is formed over the exposed surfaces of the hydrogel. In some embodiments, when the provided methods are carried out on hydrogel microparticles, the result is in situ formation of hydrogel microparticles surrounded by an siloxane shell. The provided methods are in contrast to bulk modification methods such as co-polymerization or blending of a hydrogel with hydrophobic compounds since the provided methods retain the intrinsic properties of the hydrogel or microparticle core that are desirable for optimal loading of the active ingredient. For example, the methods provided herein allow for preparation of a water-containing hydrogel or microparticle that has a hydrophobic surface coating but that retains the hydrophilic properties that are desired for optimal loading of the active ingredient to the hydrogel or microparticle.

In various embodiments, provided are methods for the preparation of siloxane surface-modified hydrogels, hydrogel pastes, and hydrogel microparticles, comprising: treating a hydrogel or hydrogel microparticles with Component (A), at least one amino-functional organosilicon compound, to form at least one siloxane-coated surface on the hydrogel or hydrogel microparticles; wherein the hydrogel or hydrogel microparticles comprise Component (B), at least one organic polymer comprising amine-reactive groups selected from carboxy-functional groups, sulfonic acid-functional groups, epoxy groups, or combinations thereof the polymer being compatible with water, alcohols, or combinations thereof; and wherein the hydrogel comprises Component (C), at least one absorbable solvent selected from water, alcohols, and combinations thereof; or the hydrogel microparticles are treated in the presence of Component (C). In some embodiments, the hydrogel or hydrogel microparticles optionally comprise or are optionally treated with one or more of Component (D), at least one active ingredient; Component (E), at least one surfactant; Component (F), at least one free-radical polymerizable compound; and Component (G), at least one organoborane free radical initiator.

In various embodiments, provided are siloxane surface-modified hydrogel pastes and hydrogel microparticles prepared by the provided methods. Such pastes and microparticles are useful in a variety of applications, including for delivery of personal and healthcare active ingredients, and delivery of agricultural active ingredients.

### Hydrogels and Hydrogel Microparticles

According to various embodiments, provided are methods of modifying surfaces of hydrogels and hydrogel microparticles. Known hydrogels and hydrogel microparticles may be surface modified by the provided methods. Additionally, methods of preparing the unmodified hydrogels and hydrogel microparticles are also known and vary depending upon their nature and use. While good results have been obtained with the use of Carbopol® polyacrylic acid gels and microparticles, one of skill in the art will appreciate that the methods and compositions described herein are not limited to such gels and microparticles.

In some embodiments, hydrogel microparticles used for the provided methods may have any shape (i.e., spherical or irregular) or size. The microparticles used may be formed directly or from the shearing or pulverizing of a gel monolith. Non-limiting examples of suitably sized microparticles include those having an average particle size of from about 0.1 μm to about 100 μm.

### Component (A), Amino-Functional Organosilicon Compounds

Generally, Component (A) comprises at least one amino-functional organosilicon compound. Said organosilicon compounds may be linear, cyclic, branched, hyperbranched or resinous. In some embodiments, Component (A) may comprise organosilicon compounds having formulae selected from:

R¹₃SiO(R¹₂SiO)ₐ(R¹R²SiO)_{b}SiR¹₃; (1)

wherein a has a value of zero to 20,000 and b has a value of 1 to 20,000; and wherein each R¹ group is independently a hydrogen, halogen, or a monovalent organic group, and each R² group is independently an amine-containing group;

R³₂R⁴SiO(R³₂SiO)_{c}(R³R⁴SiO)_{d}SiR³₂R⁴; (2)

wherein c has a value of zero to 20,000, and d has a value of zero to 20,000; and wherein each R³ is independently a hydrogen, halogen, or a monovalent organic group, and each R⁴ group is independently an amine-containing group.

Suitable R¹ and R³ groups include, but are not limited to, organic groups (linear and/or branched) such as alkyl groups, haloalkyl groups, alkenyl groups, alkynyl groups, aromatic groups, acrylate functional groups, and methacrylate functional groups; and other organic functional groups such as ether groups, cyanate ester groups, ester groups, carboxylate salt groups, mercapto groups, sulfide groups, azide groups, phosphonate groups, phosphine groups, masked isocyano groups, and hydroxyl groups. Examples of such groups include, but are not limited to, alkyl groups such as methyl, ethyl, propyl, isopropyl, n-butyl, s-butyl, and t-butyl groups, acrylate functional groups such as acryloyloxypropyl groups and methacryloyloxypropyl groups; alkenyl groups such as vinyl, allyl, and butenyl groups; alkynyl groups such as ethynyl and propynyl groups; aromatic groups such as phenyl, tolyl, and xylyl groups; cyanoalkyl groups such as cyanoethyl and cyanopropyl groups; halogenated hydrocarbon groups such as 3,3,3-trifluoropropyl, 3-chloropropyl, dichlorophenyl, and 6,6,6,5,5,4,4,3,3-nonafluorohexyl groups; alkenyloxypoly(oxyalkyene) groups such as allyloxy(polyoxyethylene), allyloxypoly(oxypropylene), and allyloxy-poly(oxypropylene)-co-poly(oxyethylene) groups; alkyloxypoly(oxyalkyene) groups such as propyloxy(polyoxyethylene), propyloxypoly(oxypropylene), and propyloxy-poly(oxypropylene)-co-poly(oxyethylene) groups; halogen substituted alkyloxypoly(oxyalkyene) groups such as perfluoropropyloxy(polyoxyethylene), perfluoropropyloxypoly(oxypropylene), and perfluoropropyloxy-poly(oxypropylene)-co-poly(oxyethylene) groups; alkoxy groups such as methoxy, ethoxy, n-propoxy, isopropoxy, n-butoxy, and ethylhexyloxy groups; aminoalkyl groups such as 3-aminopropyl, 6-aminohexyl, 1 1-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole groups; epoxyalkyl groups such as 3-glycidoxypropyl, 2-(3,4,-epoxycyclohexyl)ethyl, and 5,6-epoxyhexyl groups; ester functional groups such as actetoxyethyl and benzoyloxypropyl groups; hydroxy functional groups such as hydroxy and 2-hydroxyethyl groups; masked isocyanate functional groups such as propyl-t-butylcarbamate, and propylethylcarbamate groups; aldehyde functional groups such as undecanal and butyraldehyde groups; anhydride functional groups such as 3-propyl succinic anhydride and 3-propyl maleic anhydride groups; and metal salts of carboxylic acids such as the zinc, sodium, or potassium salts of 3-carboxypropyl and 2-carboxyethyl.

Suitable R² and R⁴ groups include, but are not limited to, monovalent amine groups such as 3-aminopropyl, 2-aminoethyl, aminomethyl, 6-aminohexyl, 1 1-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole groups. In some embodiments, R² and R⁴ may be independently selected from tertiary amine groups, such as bis(2-hydroxyethyl)-3-aminopropyl, N,N-dimethyl-3-aminopropyl, N,N-diethyl-3-aminopropyl, and N,N-diethylaminomethyl. In some embodiments, R² and R⁴ may be independently selected from aminoalkyl groups having the formula R′-(NH-A′)_{q}—NH-A-, wherein A and A′ are each independently a linear or branched alkylene group having 1 to 6 carbon atoms and optionally containing an ether linkage; q=0-4; R′ is hydrogen or an alkyl or hydroxyalkyl group having 1 to 4 carbon atoms. Examples of such aminoalkyl groups include, but are not limited to, —(CH₂)₃NH₂, —(CH₂)₄NH₂, —(CH₂)₃NH(CH₂)₂NH₂, —CH₂CH(CH₃)CH₂NH(CH₂)₂NH₂, —(CH₂)₃NHCH₂CH₂NH(CH₂)₂NH₂, —CH₂CH(CH₃)CH₂NH(CH₂)₃NH₂, —CH2CHMeCH2NH(CH2)2NHCO(CH2)3 OH, —(CH₂)₃NH(CH₂)₄NH₂, and —(CH₂)₃—O—(CH₂)₂NH₂.

Certain combinations of R¹ and/or R³ groups with R² and/or R⁴ groups may result in reduced stability of Component (A) because of reactivity of the amine groups in R² and/or R⁴ with the organic groups of R¹ and/or R³. However, stability can be improved by selection of less reactive amine groups for R² and/or R⁴. For example, in the cases where R¹ and/or R³ is a halogen, an acrylate or methacrylate functional group, an epoxy, an aldehyde, a masked isocyanate, or an anhydride functional group, stability may be improved by selection of R² and/or R⁴ groups from a tertiary amine functional group or a sterically hindered amine.

In some embodiments, Component (A) may comprise siloxane resins having structural units of organopolysiloxanes independently selected from:

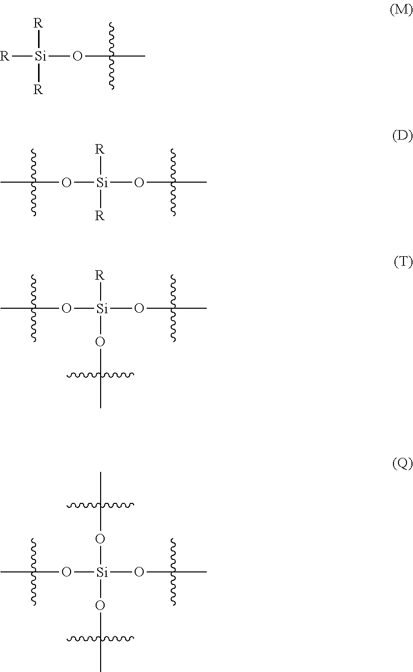

wherein M represents a monofunctional unit R₃SiO_{1/2}; D represents a difunctional unit R₂SiO_{2/2}; T represents a trifunctional unit RSiO_{3/2}; and Q represents a tetrafunctional unit SiO_{4/2}, where “R” represents any suitable functional group.

In some embodiments, Component (A) may comprise a siloxane resin selected from MQ resins having R⁵₃SiO_{1/2} units and SiO_{4/2} units; TD resins having R⁵SiO_{3/2} units and R⁵₂SiO_{2/2} units; MT resins having R⁵₃SiO_{1/2} units and R⁵SiO_{3/2} units; MTD resins having R⁵₃SiO_{1/2} units, R⁵SiO_{3/2} units, and R⁵₂SiO_{2/2} units, and combinations thereof; wherein each R⁵ group is independently a monovalent organic group having from 1-20 carbon atoms. In some embodiments, R⁵ has from 1-10 carbon atoms. In some embodiments, at least one R⁵ group is an amine-containing group.

Suitable examples of R⁵ include, but are not limited to, monovalent amine groups such as 3-aminopropyl, 2-aminoethyl, aminomethyl, 6-aminohexyl, 1 1-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole groups. In some embodiments, R⁵ may be selected from tertiary amine groups, such as bis(2-hydroxyethyl)-3-aminopropyl, N,N-dimethyl-3-aminopropyl, N,N-diethyl-3-aminopropyl, and N,N-diethylaminomethyl. In some embodiments, R⁵ may be selected from aminoalkyl groups having the formula R′-(NH-A′)_{q}—NH-A-, wherein A and A′ are each independently a linear or branched alkylene group having 1 to 6 carbon atoms and optionally containing an ether linkage; q=0-4; R′ is hydrogen or an alkyl or hydroxyalkyl group having 1 to 4 carbon atoms. Examples of such aminoalkyl groups include, but are not limited to, —(CH₂)₃NH₂, —(CH₂)₄NH₂, —(CH₂)₃NH(CH₂)₂NH₂, —CH₂CH(CH₃)CH₂NH(CH₂)₂NH₂, —(CH₂)₃NHCH₂CH₂NH(CH₂)₂NH₂, —CH₂CH(CH₃)CH₂NH(CH₂)₃NH₂, —(CH₂)₃NH(CH₂)₄NH₂, and —(CH₂)₃—O—(CH₂)₂NH₂.

In some embodiments, Component (A) may be selected from poly[dimethyl, methyl (aminoethylaminoisobutyl)] siloxane, poly[dimethyl, methyl (aminoethylaminopropyl)] siloxane, poly[(dimethyl, methylaminopropyl)] siloxane, aminopropyl-terminated polydimethylsiloxane, amino ethylaminopropyl-terminated polydimethylsiloxane, and aminoethylaminoisobutyl-terminated polydimethylsiloxane.

### Optional Solvent for Component (A)

In some embodiments, the preparation of surface-modified hydrogels, hydrogel pastes, and hydrogel microparticles involves optionally treating a hydrogel or hydrogel microparticle in the presence of a suitable solvent for Component (A). In some embodiments, suitable solvents for Component (A) may be selected from water-immiscible silicones; organic compounds; and “ecologically-friendly” solvents, such as ionic liquids and supercritical fluids; and mixtures thereof. Examples of suitable solvents include, but are not limited to, linear, branched, hyperbranched and cyclic organosiloxane fluids, such as hexamethyldisiloxane, octamethyltrisiloxane, decamethyltetrasiloxane, and trimethylsilyl-terminated polydimethylsiloxane fluids having a viscosity of less than 1000 cP at 25° C., or a mixture thereof; capryllylmethyl trisiloxane; octamethylcyclotetrasiloxane; decamethylcyclopentasiloxane; and higher cyclosiloxanes and mixtures thereof. In some embodiments, trimethylsilyl-terminated polydimethylsiloxane fluids suitable as a solvent for Component (A) have a viscosity of from about 0.5 to about 100 cP at 25° C. Other suitable solvents include, but are not limited to, organic solvents immiscible with water, such as pentane, hexane, heptane, octane, cyclohexane, toluene, xylenes, ethyl acetate. Further examples of suitable solvents for Component (A) include, but are not limited to, organic oils such as isododecane, isohexadecane, isodecylneopentanoate, isononyl isononanoate, isoparaffin, isoalkane, and ionic liquids including, 1-ethenyl-3-ethyl-imidazolium hexafluorophosphate and tetrapropyl-ammonium tetracyanoborate, and supercriticial fluids such as supercritical carbon dioxide.

### Component (B), Organic Polymer Comprising Amine-Reactive Groups

Generally, Component (B) comprises at least one organic polymer comprising amine-reactive groups. The polymer may be homopolymeric, heteropolymeric (including, but not limited to, cross-polymers or co-polymers of any co-monomer distribution), and may be linear, branched, hyperbranched, dendrimeric, or crosslinked to any extent. In some embodiments, amine-reactive groups are selected from carboxy-functional groups, sulfonic acid-functional groups, epoxy groups, or combinations thereof. In various embodiments, Component (B) is water-compatible. In some embodiments, Component (B) has at least 5 mol % of amine-reactive groups. In some embodiments, Component (B) has from about 5 mol % to about 10 mol % of amine-reactive groups. In some embodiments, Component (B) has at least 10 mol % of amine-reactive groups.

In some embodiments, Component (B) may be selected from a carboxy-functional organic polymer, an anhydride-functional organic polymer, and an epoxy-functional organic polymer. Examples of suitable polymers include, but are not limited to polyacrylic acid, poly(meth)acrylic acid, salts of polyacrylic acid, salts of poly(methacrylic acid), poly(2-hydroxyethyl methacrylate), polylactic acid, polyglycolic acid, polyanhydrides such as poly(methacrylic) anhydride, poly(acrylic) anhydride, polysebasic anhydride, poly(hyaluronic acid), and hyaluronic acid containing polymers and copolymers, and combinations thereof. The polymers may also be copolymers comprised of water-compatible monomeric units and amine-reactive monomeric units, such as a poly(ethylhexylmethacrylate)-polyacrylic acid copolymer, or a polyvinylalcohol—polyacrylic acid copolymer. Good results have been obtained with selection of Component (B) from polyacrylic acid, poly(meth)acrylic acid, and partially crosslinked polyacrylic acid homopolymers, ionomers and copolymers such as Carbopol® ETD 2020, Carbopol® Ultrez 20, and Carbopol® ETD 2050.

### Component (C), Absorbable Solvent

Generally, Component (C) is at least one solvent that is absorbable by the hydrogel or hydrogel microparticle. In some embodiments, the absorbable solvent may be selected from water, water compatible alcohols, diols, polyols, and combinations thereof. Examples of suitable alcohols include, but are not limited to, methanol, ethanol, isopropyl alcohol, ethylene glycol, polyethylene glycol and combinations thereof. In some embodiments, the absorbable solvent may be a combination of one or more water-compatible alcohols with water.

### Optional Component (D), Active Ingredient

Generally, optional Component (D) comprises at least one active ingredient selected from personal care or healthcare active ingredients, or from agricultural active ingredients. In some embodiments, Component (D) comprises at least one active ingredient that can be added to the hydrogel or hydrogel microparticle for in situ encapsulation. In some embodiments, Component (D) may be added during the making of the hydrogel or hydrogel microparticle (pre-load method), added after formation of the hydrogel or hydrogel microparticle (post-load method), or added after formation of the surface-modified hydrogel or hydrogel microparticle (post-modification method). In some embodiments, the active ingredient suspended in the hydrogel or hydrogel microparticle can be, but is not required to be, in particulate form. By careful selection of the other components of the hydrogel or hydrogel microparticle, properties may be controlled to allow a desirable mechanism of release of the active ingredient. Examples of release mechanisms include extraction, dissolution, swelling, melting, softening, degradation, abrading, squeezing or cracking via thermal, mechanical, or chemical or radiation-induced stress.

The amount of Component (D) present in the hydrogel or hydrogel microparticle may vary, but in some embodiments ranges from about 0% to about 50% (by weight), alternatively from about 1% to about 25% (by weight), alternatively from about 1% to about 10% (by weight), based on the amount by total weight of components.

As used herein, a “personal care or healthcare active ingredient” means any compound or mixtures of compounds that may be used as additives in personal care formulations that are typically added for the purpose of providing a cosmetic and/or aesthetic benefit, a pharmaceutical or medical benefit, a pharmacological activity or other direct effect in the diagnosis, cure, mitigation, treatment, or prevention of disease, or to affect the structure or any function of the body of a human or other animals. Thus, “personal care and healthcare active ingredient” includes, but is not limited to, an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

In some embodiments, active ingredients suitable for Component (D) include both fat or oil-soluble vitamins, as well as water-soluble vitamins. Oil-soluble vitamins useful as Component (D) include, but are not limited to, Vitamin A1, RETINOL, C2-C18 esters of RETINOL, Vitamin E, TOCOPHEROL, esters of Vitamin E, and mixtures thereof. RETINOL includes trans-RETINOL, 1,3-cis-RETINOL, 11-cis-RETINOL, 9-cis-RETINOL, and 3,4-didehydro-RETINOL. It should be noted that RETINOL is an International Nomenclature Cosmetic Ingredient Name (INCI) designated by The Cosmetic, Toiletry, and Fragrance Association (CTFA), Washington D.C., for Vitamin A. Other suitable vitamins and the INCI names for the vitamins considered included herein are RETINYL ACETATE, RETINYL PALMITATE, RETINYL PROPIONATE, a-TOCOPHEROL, TOCOPHERSOLAN, TOCOPHERYL ACETATE, TOCOPHERYL LINOLEATE, TOCOPHERYL NICOTINATE, and TOCOPHERYL SUCCINATE.

Water-soluble vitamins useful as Component (D) include, but are not limited to, Vitamin C, Vitamin B1, Vitamin B2, Vitamin B6, Vitamin B12, niacin, folic acid, biotin, and pantothenic acid. Other suitable water-soluble vitamins and the INCI names for the vitamins considered included herein are ASCORBYL DIPALMITATE, ASCORBYL METHYLSILANOL PECTINATE, ASCORBYL PALMITATE, and ASCORBYL STEARATE.

Some examples of commercially available products suitable for use as Component (D) are Vitamin A Acetate and Vitamin C, both products of Fluka Chemie AG, Buchs, Switzerland; COVI-OX T-50, a Vitamin E product of Henkel Corporation, La Grange, Ill.; COVI-OX T-70, another Vitamin E product of Henkel Corporation, La Grange, Ill.; and Vitamin E Acetate, a product of Roche Vitamins & Fine Chemicals, Nutley, N.J.

In some embodiments, the personal care or healthcare active ingredient used as Component (D) can be a water-soluble or an oil-soluble active drug ingredient. Representative examples of some suitable water-soluble active drug ingredients which can be used are hydrocortisone, ketoprofen, timolol, pilocarpine, adriamycin, mitomycin C, morphine, hydromorphone, diltiazem, theophylline, doxorubicin, daunorubicin, heparin, penicillin G, carbenicillin, cephalothin, cefoxitin, cefotaxime, 5-fluorouracil, cytarabine, 6-azauridine, 6-thioguanine, vinblastine, vincristine, bleomycin sulfate, aurothioglucose, suramin, and mebendazole.

Representative examples of some suitable oil-soluble active drug ingredients which can be used as Component (D) are clonidine, scopolamine, propranolol, phenylpropanolamine hydrochloride, ouabain, atropine, haloperidol, isosorbide, nitroglycerin, ibuprofen, ubiquinones, indomethacin, prostaglandins, naproxen, salbutamol, guanabenz, labetalol, pheniramine, metrifonate, and steroids.

Considered to be included herein as active drug ingredients for purposes of the present invention are antiacne agents such as benzoyl peroxide and tretinoin; antibacterial agents such as chlorohexadiene gluconate; antifungal agents such as miconazole nitrate; anti-inflammatory agents; corticosteroidal drugs; non-steroidal anti-inflammatory agents such as diclofenac; antipsoriasis agents such as clobetasol propionate; anaesthetic agents such as lidocaine; antipruritic agents; and antidermatitis agents.

In some embodiments, Component (D) can also be a protein, such as an enzyme. Enzymes include, but are not limited to, commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include, but are not limited to, proteases (bacterial, fungal, acid, neutral or alkaline), amylases (alpha or beta), lipases, mannanases, cellulases, collagenases and mixtures thereof.

In some embodiments, Component (D) may be a sunscreen agent. The sunscreen agent can be selected from any sunscreen agent known in the art to protect skin from the harmful effects of exposure to sunlight. The sunscreen can be an organic compound, an inorganic compound, or mixtures thereof. Thus, representative non limiting examples that can be used as the sunscreen agent include; Aminobenzoic Acid, Cinoxate, Diethanolamine Methoxycinnamate, Digalloyl Trioleate, Dioxybenzone, Ethyl 4-[bis(Hydroxypropyl)] Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate 0, Phenylbenzimidazole Sulfonic Acid, Red Petrolatum, Sulisobenzone, Titanium Dioxide, and Trolamine Salicylate.

The organic sunscreen compound is typically chosen from an organic compound that absorbs ultraviolet (UV) light. Some examples of UV light absorbing compounds are Acetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycin namate, Dibenzoxazol Naphthalene, Di-t-Butyl Hydroxybenzyl idene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, isopropylbenzyl Salicylate, Isopropyl Dibenzolylmethane, isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, TriPABA Panthenol, Urocanic Acid, and VA/Crotonates/Methacryloxybenzophenone copolymer.

Alternatively, the sunscreen agent is a cinnamate based organic compound, or alternatively, the sunscreen agent is octyl methoxycinnamate, such as Uvinul® MC 80 an ester of para-methoxycinnamic acid and 2-ethylhexanol.

In some embodiments, Component (D) may be any perfume or fragrance active ingredient commonly used in industry. These compositions typically belong to a variety of chemical classes, as varied as alcohols, aldehydes, ketones, esters, ethers, acetates, nitrites, terpenic hydrocarbons, heterocyclic nitrogen or sulphur containing compounds, as well as essential oils of natural or synthetic origin. Many of these perfume active ingredients are described in detail in standard textbook references such as Perfume and Flavor Chemicals, 1969, S. Arctander, Montclair, N.J..

Fragrance active ingredients may be exemplified by, but not limited to, perfume ketones and perfume aldehydes. Illustrative of the perfume ketones are buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-lonone, Beta-lonone, Gamma-Methyl so-called lonone, Fleuramone, Dihydroj asmone, Cis-Jasmone, lso-E-Super, Methyl-Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethylCyclohexanone Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane, 6,7-Dihydro-1,1,2,3,3-Pentamethyl-4(5H)-indanone 4-Damascol, Dulcinyl or Cassione, Gelsone, Hexylon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, and Tetrameran.

Perfume ketones may be, but are not required to be, selected for odor character from Alpha Damascone, Delta Damascone, Iso Damascone, Carvone, Gamma-Methyl-lonone, lso-E-Super, 2,4,4,7-Tetramethyl-oct-6-en-3-one, Benzyl Acetone, Beta Damascone, Damascenone, methyl dihydrojasmonate, methyl cedrylone, and mixtures thereof.

A perfume aldehyde may be, but is not required to be, selected for its odor character from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cm namic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, paratolylacetaldehyde; 4-methylphenylacetaldehyde, 2-methyl-4-(2,6,6-trimethyl-1-cyclohexen-1-yl)-2-butena I, ortho-methoxycinnamic aldehyde, 3,5,6-trimethyl-3-cyclohex ene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-aI), hexahydro-4,7-methanoindan-1-carboxaldehyde, 2-methyl octanal, alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2. 1]-hept-5-ene-2-carbaldehyde, 9-decenal, 3-methyl-5-phenyl-1-pentanal, methylnonyl acetaldehyde, hexanal, trans-2-hexenal, 1-p-menthene-q-carboxaldehyde and mixtures thereof.

Aldehydes may also be, but are not required to be, selected for their odor character from 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, hexanal, trans-2-hexenal, and mixture thereof.

In the above list of perfume active ingredients, some are commercial names conventionally known to one skilled in the art, and also includes isomers. Such isomers are also suitable for use in the present invention.

In some embodiments, Component (D) may be one or more plant extracts. Examples of these components are as follows: Ashitaba extract, avocado extract, hydrangea extract, Althea extract, Arnica extract, aloe extract, apricot extract, apricot kernel extract, Ginkgo Biloba extract, fennel extract, turmeric[Curcuma] extract, oolong tea extract, rose fruit extract, Echinacea extract, Scutellaria root extract, Phellodendro bark extract, Japanese Coptis extract. Barley extract, Hyperium extract, White Nettle extract, Watercress extract, Orange extract, Dehydrated saltwater, seaweed extract, hydrolyzed elastin, hydrolyzed wheat powder, hydrolyzed silk, Chamomile extract, Carrot extract, Artemisia extract, Glycyrrhiza extract, hibiscus tea extract, Pyracantha Fortuneana Fruit extract, Kiwi extract, Cinchona extract, cucumber extract, guanocine. Gardenia extract, Sasa Albo-marginata extract, Sophora root extract, Walnut extract, Grapefruit extract, Clematis extract, Chlorella extract, mulberry extract, Gentiana extract, black tea extract, yeast extract, burdock extract, rice bran ferment extract, rice germ oil, comfrey extract, collagen, cowberry extract, Gardenia extract, Asiasarum Root extract, Family of Bupleurum extract, Salvia extract, Saponaria extract, Bamboo extract, Crataegus fruit extract, Zanthoxylum fruit extract, shiitake extract, Rehmannia root extract, gromwell extract, Perilla extract, linden extract, Filipendula extract, peony extract, Calamus Root extract, white birch extract, Horsetail extract, Hedera Helix(Ivy) extract, hawthorn extract, Sambucus nigra extract, Achillea millefolium extract, Mentha piperita extract, sage extract, mallow extract, Cnidium officinale Root extract, Japanese green gentian extract, soybean extract, jujube extract, thyme extract, tea extract, clove extract, Gramineae imperata cyrillo extract, Citrus unshiu peel extract Japanese Angellica Root extract, Calendula extract. Peach Kernel extract, Bitter orange peel extract, Houttuyna cordata extract, tomato extract, natto extract, Ginseng extract, Green tea extract (camelliea sinesis), garlic extract, wild rose extract, hibiscus extract, Ophiopogon tuber extract, Nelumbo nucifera extract, parsley extract, honey, hamamelis extract, Parietaria extract, Isodonis herba extract, bisabolol extract, Loquat extract, coltsfoot extract, butterbur extract, Porid cocos wolf extract, extract of butcher's broom, grape extract, propolis extract, luffa extract, safflower extract, peppermint extract, linden tree extract, Paeonia extract, hop extract, pine tree extract, horse chestnut extract, Mizu-bashou extract, Mukurossi peel extract, Melissa extract, peach extract, cornflower extract, eucalyptus extract, saxifrage extract, citron extract, coix extract, mugwort extract, lavender extract, apple extract, lettuce extract, lemon extract, Chinese milk vetch extract, rose extract, rosemary extract, Roman Chamomile extract, and royal jelly extract.

In some embodiments, Component (D) comprises at least one agricultural active ingredient. As used herein, an “agricultural active ingredient” means any compound or mixtures of compounds that may be additives in formulations that are typically added for the purpose of treating plants.

Examples of suitable agricultural active ingredients include, but are not limited to, 2-phenylphenol; 8-hydroxyquinoline sulfate; AC 382042; Ampelomyces quisqualis; Azaconazole; Azoxystrobin; Bacillus subtilis; Benalaxyl; Benomyl; Biphenyl; Bitertanol; Blasticidin-S; Bordeaux mixture; Borax; Bromuconazole; Bupirimate; Calboxin; calcium polysulfide; Captafol; Captan; Carbendazim; Carpropanmid (KTU 3616); CGA 279202; Chinomethionat; Chlorothalonil; Chlozolinate; copper hydroxide; copper naphthenate; copper oxychloride; copper sulfate; cuprous oxide; Cymoxanil; Cyproconazole; Cyprodinil; Dazomet; Debacarb; Dichlofluanid; Dichlomezine; Dichlorophen; Diclocymet; Dicloran; Diethofencarb; Difenoconazole; Difenzoquat; Difenzoquat metilsulfate; Diflumetorim; Dimethirimol; Dimethomorph; Diniconazole; Diniconazole-M; Dinobuton; Dinocap; diphnenylamine; Dithianon; Dodemorph; Dodemorph acetate; Dodine; Dodine free base; Edifenphos; Epoxiconazole (BAS 480F); Ethasulfocarb; Ethirimol; Etridiazole; Famoxadone; Fenamidone; Fenarimol; Fenbuconazole; Fenfin; Fenfuram; Fenhexamid; Fenpiclonil; Fenpropidin; Fenpropimorph; Fentin acetate; Fentin hydroxide; Ferbam; Ferimzone; Fluazinam; Fludioxonil; Fluoroimide; Fluquinconazole; Flusilazole; Flusulfamide; Flutolanil; Flutriafol; Folpet; formaldehyde; Fosetyl; Fosetyl-aluminum; Fuberidazole; Furalaxyl; Fusarium oxysporum; Gliocladium vixens; Guazatine; Guazatine acetates; GY-81; hexachlorobenzene; Hexaconazole; Hymexazol; ICIA0858; IKF-916; Imazalil; Imazalil sulfate; Imibenconazole; Iminoctadine; Iminoctadine triacetate; Iminoctadine tris[Albesilate]; Ipconazole; Iprobenfos; Iprodione; Iprovalicarb; Kasugamycin; Kasugamycin hydrochloride hydrate; Kresoxim-methyl; Mancopper; Mancozeb; Maneb; Mepanipyrim; Mepronil; mercuric chloride; mercuric oxide; mercurous chloride; Metalaxyl; Metalaxyl-M; Metam; Metam-sodium; Metconazole; Methasulfocarb; methyl isothiocyanate; Metiram; Metominostrobin (SSF-126); MON65500; Myclotbutanil; Nabam; naphthenic acid; Natamycin; nickel bis(dimethyldithiocarbamate); Nitrothal-isopropyl; Nuarimol; Octhilinone; Ofurace; oleic acid (fatty acids); Oxadixyl; Oxine-copper; Oxycarboxin; Penconazole; Pencycuron; Pentachlorophenol; pentachlorophenyl laurate; Perfurazoate; phenylmercury acetate; Phlebiopsis gigantea; Phthalide; Piperalin; polyoxin B; polyoxins; Polyoxorim; potassium hydroxyquinoline sulfate; Probenazole; Prochloraz; Procymidone; Propamocarb; Propamocarb Hydrochloride; Propiconazole; Propineb; Pyrazophos; Pyributicarb; Pyrifenox; Pyrimethanil; Pyroquilon; Quinoxyfen; Quintozene; RH-7281; sec-butylamine; sodium 2-phenylphenoxide; sodium pentachlorophenoxide; Spiroxamine (KWG 4168); Streptomyces griseoviridis; sulfur; tar oils; Tebuconazole; Tecnazene; Tetraconazole; Thiabendazole; Thifluzamide; Thiophanate-methyl; Thiram; Tolclofos-methyl; Tolylfluanid; Triadimefon; Triadimenol; Triazoxide; Trichoderma harzianum; Tricyclazole; Tridemorph; Triflumizole; Triforine; Triticonzole; Validamycin; vinclozolin; zinc naphthenate; Zineb; Ziram; the compounds having the chemical name methyl (E,E)-2-(2-(1-(1-(2-pyridyl)propyloxyimino)-1-cyclopropylmethyloxymethyl)phenyl)-3-ethoxypropenoate and 3-(3,5-dichlorophenyl)-4-chloropyrazole; 2-anilino-4-methyl-6-cyclopropylpyrimidine; 2′,6′-dibromo-2-methyl-4′-trifluoromethoxy-4′-trifluoromethyl-1,3-thiazol-5-carboxanilide; 2,6-dichloro-N-(4-trifluoromethylbenzyl)benzamide; (E)-2-methoximino-N-methyl-2-(2-phenoxyphenyl)-acetamide; 8-hydroxyquinoline sulfate; methyl (E)-2-{2-[6-(2-cyanophenoxy)-pyrimidin-4-yloxy]phenyl}-3-methoxyacrylate; methyl (E)-methoximino[alpha-(o-tolyloxy)-o-tolyl]acetate; 2-phenylphenol (OPP), ampropylfos, anilazine, benodanil, binapacryl, buthiobate, carboxin, quinomethionate, chloroneb, chloropicrin, cufraneb, cyprofuram, diclobutrazole, dichlofluanid, dipyrithione, ditalimfos, drazoxolone, fenitropan, fthalide, furmecyclox, isoprothiolane, methfuroxam, metsulfovax, oxamocarb, pefurazoate, penconazole, phosdiphen, pimaricin, tecloftalam, thicyofen, trichlamide, trifloxystrobin, 2-[2-(1-chlorocyclopropyl)-3-(2-chlorophenyl)-2-hydroxypropyl]-2,4-dihydro-[1,2,4]-triazole-3-thione 3-(1-[2-(4-[2-chlorophenoxy)-5-fluoropyrimid-6-yloxy)-phenyl]-1-(methoximino)-methyl)-5,6-dihydro-1,4,2-dioxazine and N-methyl-2-(2-[6-(3-chloro-2-methylphenoxy)-5-fluoropyrmid-4-yloxy]phenyl)-2-methoximinoacetamide.

Examples of suitable agricultural active ingredients also include, but are not limited to, Abamectin; Acephate; Acetamiprid; oleic acid; Acrinathrin; Aldicarb; Alanycarb; Allethrin [(1R) isomers]; α-Cypermethrin; Amitraz; Avermectin B1 and its derivatives, Azadirachtin; Azamethiphos; Azinphos-ethyl; Azinphosmethyl; Bacillus thurigiensi; Bendiocarb; Benfuracarb; Bensultap; β-cyfluthrin; β-cypermethrin; Bifenazate; Bifenthrin; Bioallathrin; Bioallethrin (S-cyclopentenyl isomer); Bioresmethrin; Borax; Buprofezin; Butocarboxim; Butoxycarboxim; piperonyl butoxide; Cadusafos; Carbaryl; Carbofuran; Carbosulfan; Cartap; Cartap hydrochloride; Chordane; Chlorethoxyfos; Chlorfenapyr; Chlorfenvirnphos; Chlorfluazuron; Chlormephos; Chloropicrin; Chlorpyrifos; Chlorpyrifos-methyl; mercurous chloride; Coumaphos; Cryolite; Cryomazine; Cyanophos; calcium cyanide; sodium cyanide; Cycloprothrin; Cyfluthrin; Cyhalothrin; cypermethrin; cyphenothrin [(1R) transisomers]; Dazomet; DDT; Deltamethrin; Demeton-5-methyl; Diafenthiuron; Diazinon; ethylene dibromide; ethylene dichloride; Dichlorvos; Dicofol; Dicrotophos; Diflubenzuron; Dimethoate; Dimethylvinphos; Diofenolan; Disulfoton; DNOC; DPX-JW062 and DP; Empenthrin [(EZ)-(1R) isomers]; Endosulfan; ENT 8184; EPN; Esfenvalerate; Ethiofencarb; Ethion; Ethiprole having the chemical name 5-amino-3-cyano-1-(2,6-dichloro-4-trifluoromethylphenyl)-4-ethylsulfinylpyrazole; Ethoprophos; Etofenprox; Etoxazole; Etrimfos; Famphur; Fenamiphos; Fenitrothion; Fenobucarb; Fenoxycarb; Fenpropathrin; Fenthion; Fenvalerate; Fipronil and the compounds of the arylpyrazole family; Flucycloxuron; Flucythrinate; Flufenoxuron; Flufenprox; Flumethrin; Fluofenprox; sodium fluoride; sulfuryl fluoride; Fonofos; Formetanate; Formetanate hydrochloride; Formothion; Furathiocarb; Gamma-HCH; GY-81; Halofenozide; Heptachlor; Heptenophos; Hexaflumuron; sodium hexafluorosilicate; tar oils; petroleum oils; Hydramethylnon; hydrogen cyanide; Hydroprene; Imidacloprid; Imiprothrin; Indoxacarb; Isazofos; Isofenphos; Isoprocarb; Methyl isothiocyanal; Isoxathion; lambda-Cyhalothrin; pentachlorophenyl laurate; Lufenuron; Malathion; MB-599; Mecarbam; Methacrifos; Methamidophos; Methidathion; Methiocarb; Methomyl; Methoprene; Methoxychlor; Metolcarb; Mevinphos; Milbemectin and its derivatives; Monocrotophos; Naled; nicotine; Nitenpyram; Nithiazine; Novaluron; Omethoate; Oxamyl; Oxydemeton-methyl; Paecilomyces fumosoroseus; Parathion; Parathion-methyl; pentachlorophenol; sodium pentachlorophenoxide; Permethrin; Penothrin [(1R)-trans-isomers]; Phenthoate; Phorate; Phosalone; Phosmet; Phosphamidon; phosphine; aluminum phosphide; magnesium phosphide; zinc phosphide; Phoxim; Pirimicarb; Pirimiphos-ethyl; Pirimiphos-methyl; calcium polysulfide; Prallethrin; Profenfos; Propaphos; Propetamphos; Propoxur; Prothiofos; Pyraclofos; pyrethrins (chrysanthemates, pyrethrates, pyrethrum; Pyretrozine; Pyridaben; Pyridaphenthion; Pyrimidifen; Pyriproxyfen; Quinalphos; Resmethrin; RH-2485; Rotenone; RU 15525; Silafluofen; Sulcofuron-sodium; Sulfotep; sulfuramide; Sulprofos; Ta-fluvalinate; Tebufenozide; Tebupirimfos; Teflubenzuron; Tefluthrin; Temephos; Terbufos; Tetrachlorvinphos; Tetramethrin; Tetramethrin [(1R) isomers]; θ-cypermethrin; Thiametoxam; Thiocyclam; Thiocyclam hydrogen oxalate; Thiodicarb; Thiofanox; Thiometon; Tralomethrin; Transfluthrin; Triazamate; Triazophos; Trichlorfon; Triflumuron; Trimethacarb; Vamidothion; XDE-105; XMC; Xylylcarb; Zeta-cypermethrin; ZXI 8901; the compound whose chemical name is 3-acetyl-5-amino-1-[2,6-dichloro-4-(trifluoromethyl)phenyl]-2-methylsulfinylpyrazole; alphamethrin, AZ 60541, azinphos A, azinphos M, azocyclotin, 4-bromo-2-(4-chlorophenyl)-1-(ethoxymethyl)-5-(trifluoromethyl)-1H-pyrrole-3-carbonitrile, BPMC, brofenprox, bromophos A, bufencarb, butylpyridaben, carbophenothione, chloethocarb, N-[(6-chloro-3-pyridinyl)-methyl]-N′-cyano-N-methylethanimidamide, clocythrin, clofentezin, cyhexatin, cyromazin, clothianidin, demeton-M, demeton-S, dichlofenthion, dicliphos, diethion, dioxathion, emamectin, esfenvalerate, fenazaquin, fenbutatin oxide, fenothiocarb, fenpyrad, fenpyroximate, fluazuron, fluvalinate, fosthiazate, fubfenprox, hexythiazox, iprobenfos, ivermectin, mesulfenphos, metaldehyde, moxidectin, NC 184, oxydeprofos, promecarb, prothoate, pymetrozin, pyresmethrin, salithion, sebufos, tebufenpyrad, tebupirimiphos, terbam, thiacloprid, thiafenox, thiamethoxam, thiomethon, thuringiensin, triarathen, triazuron, trichlorfon, zetamethrin.

Examples of suitable agricultural active ingredients further include, but are not limited to, metaldehyde and methiocarb.

In addition, suitable examples of agricultural active ingredients include, but are not limited to, 2,3,6-TBA; 2,4-D; 2,4-D-2-ethylhexyl; 2,4-DB; 2,4-DB-butyl; 2,4-DB-dimethylammonium; 2,4-DB-isooctyl; 2,4-DB-potassium; 2,4-DB-sodium; 2,4-D-butotyl (2,4-D-Butotyl (2,4-D Butoxyethyl Ester)); 2,4-D-butyl; 2,4-D-dimethylammonium; 2,4-D-Diolamine; 2,4-D-isoctyl; 2,4-D-isopropyl; 2,4-D-sodium; 2,4-D-trolamine; Acetochlor; Acifluorfen; Acifluorfen-sodium; Aclonifen; Acrolein; AKH-7088; Alachlor; Alloxydim; Alloxydim-sodium; Ametryn; Amidosulfuron; Amitrole; ammonium sulfamate; Anilofos; Asulam; Asulam-sodium; Atrazine; Azafenidin; Azimsulfuron; Benazolin; Benazolin-ethyl; Benfluralin; Benfuresate; Benoxacor; Bensulfuron; Bensulfuron-methyl; Bensulide; Bentazone; Bentazone-sodium; Benofenap; Bifenox; Bilanofos; Bilanafos-sodium; Bispyribac-sodium; Borax; Bromacil; Bromobutide; Bromofenoxim; Bromoxynil; Bromoxynil-heptanoate; Bromoxynil-octanoate; Bromoxynil-potassium, Butachlor; Butamifos; Butralin; Butroxydim; butylate; Cafenstrole; Carbetamide; Carfentrazone-ethyl; Chlomethoxyfen; Chloramben; Chlorbromuron; Chloridazon; Chlorimuron; Chlorimuron-ethyl; Chloroacetic Acid; Chlorotoluron; Chlorpropham; Chlorsulfuron; Chlorthal; Chlorthal-dimethyl; Chlorthiamid; Cinmethylin; Cinosulfuron; Clethodim; Clodinafop; Clodinafop-Propargyl; Clomazone; Clomeprop; Clopyralid; Clopyralid-Olamine; Cloquintocet; Cloquintocet-Mexyl; Chloransulam-methyl; CPA; CPA-dimethylammonium; CPA-isoctyl; CPA-thioethyl; Cyanamide; Cyanazine; Cycloate; Cyclosulfamuron; Cycloxydim; Cyhalofop-butyl; Daimuron; Dalapon; Dalapon-sodium; Dazomet; Desmeduipham; Desmetryn; Dicamba; Dicamba-dimethylammonium; Dicamba-potassium; Dicamba-sodium; Dicamba-trolamine; Dichlobenil; Dichlormid; Dichlorprop; Dichlorprop-butotyl (Dichlorprop-butotyl (Dichlorpropbutoxyethyl ester)); Dichlorprop-dimethylammonium; Dichlorprop-isoctyl; Dichlorprop-P; Dichlorprop-potassium; Diclofop; Diclofop-methyl; Difenzoquat; Difenzoquat metilsulfate; Diflufenican; Diflufenzopyr (BAS 654 00 H); Dimefuron; Dimepiperate; Dimethachlor; Dimethametryn; Dimethenamid; Dimethipin; dimethylarsinic acid; Dinitramine; Dinoterb; Dinoterb acetate; Dinoterb-ammonium; Dinoterb-diolamine; Diphenamid; Diquat; Diquat dibromide; Dithiopyr; Diuron; DNOC; DSMA; Endothal; EPTC; Esprocarb; Ethalfluralin; Ethametsulfuron-methyl; Ethofumesate; Ethoxysulfuron; Etobenzanid; Fenchlorazole-ethyl; Fenclorim; Fenoxaprop-P; Fenoxaprop-P-ethyl; Fenuron; Fenuron-TCA; Ferrous Sulfate; Flamprop-M; Flamprop-M-Isopropyl; Flamprop-M-methyl; Flazasulfuron; Fluazifop; Fluazifop-butyl; Fluazifop-P; Fluazifop-P-butyl; Fluazolate; Fluchloralin; Flufenacet (BAS FOE 5043); Flumetsulam; Flumiclorac; Flumiclorac-Pentyl; Flumioxazin; Fluometuron; Fluoroglycofen; Fluoroglycofen-ethyl; Flupaxam; Flupoxam; Flupropanate; Flupropanate-sodium; Flupyrsulfuron-methyl-sodium; Flurazole; Flurenol; Flurenol-butyl; Fluridone; Fluorochloridone; Fluoroxypyr; Fluoroxypyr-2-Butoxy-1-methylethyl; Fluoroxypyr-methyl; Flurtamone; Fluthioacet-methyl; Fluxofenim; Fomesafen; Fomesafen-sodium; Fosamine; Fosamine-ammonium; Furilazole; Glyphosate; Glufosinate; Glufosinate-ammonium; Glyphosate-ammonium; Glyphosate-isopropylammonium; Glyphosate-sodium; Glyphosate-trimesium; Halosulfuron; Halosulfuron-methyl; Haloxyfop; Haloxyfop-P-methyl; Haloxyfop-etotyl; Haloxyfop-methyl; Hexazinone; Hilanafos; Imazacluin; Imazamethabenz; Imazamox; Imazapyr; Imazapyr-isopropylammonium; Imazaquin; Imazaquin-ammonium; Imazemethabenz-methyl; Imazethapyr; Imazethapyr-ammonium; Imazosulfuron; Imizapic (AC 263,222); Indanofan; Ioxynil; Ioxynil octanoate; Ioxynil-sodium; Isoproturon; Isouron; Isoxaben; Isoxaflutole; Lactofen; Laxynel octanoate; Laxynil-sodium; Lenacil; Linuron; MCPA; MCPA-butotyl; MCPA-dimethylammonium; MCPA-isoctyl; MCPA-potassium; MCPA-sodium; MCPA-thioethyl; MCPB; MCPB-ethyl; MCPB-sodium; Mecoprop; Mecoprop-P; Mefenacet; Mefenpyr-diethyl; Mefluidide; Mesulfuron-methyl; Metam; Metamitron; Metam-sodium; Metezachlor; Methabenzthiazuron; methyl isothiocyanate; methylarsonic acid; Methyldymron; Metobenzuron; Metobromuron; Metolachlor; Metosulam; Metoxuron; Metribuzin; Metsulfuron; Molinate; Monolinuron; MPB-sodium; MSMA; Napropamide; Naptalam; Naptalam-sodium; Neburon; Nicosulfuron; nonanoic acid; Norflurazon; oleic acid (fatty acids); Orbencarb; Oryzalin; Oxabetrinil; Oxadiargyl; Oxasulfuron; Oxodiazon; Oxyfluorfen; Paraquat; Paraquat Dichloride; Pebulate; Pendimethalin; Pentachlorophenol; Pentachlorophenyl Laurate; Pentanochlor; Pentoxazone; petroleum oils; Phenmedipham; Picloram; Picloram-potassium; Piperophos; Pretilachlor; Primisulfuron; Primisulfuron-methyl; Prodiamine; Prometon; Prometryn; Propachlor; Propanil; Propaquizafop; Propazine; Propham; Propisochlor; Propyzamide; Prosulfocarb; Prosulfuron; Pyraflufen-ethyl; Pyrazasulfuron; Pyrazolynate; Pyrazosulfuron-ethyl; Pyrazoxyfen; Pyribenzoxim; Pyributicarb; Pyridate; Pyriminobac-methyl; Pyrithiobac-sodium; Quinclorac; Quinmerac; Quinofolamine; Quizalofop; Quizalofop-ethyl; Quizalofop-P; Quizalofop-P-ethyl; Quizalofop-P-Tefuryl; Rimsulfuron; Sethoxydim; Siduron; Silthiopham; Simazine; Simetryn; sodium chlorate; sodium chloroacetate; sodium pentachlorophenoxide; sodium-Dimethylarsinate; Sulcotrione; Sulfentrazone; Sulfometuron; Sulfometuron-methyl; Sulfosulfuron; Sulfuric acid; tars; TCA-sodium; Tebutam; Tebuthiuron; Tepraluxydim (BAS 620H); Terbacil; Terbumeton; Terbuthylazine; Terbutryn; Thenylchlor; Thiazopyr; Thifensulfuron; Thifensulfuron-methyl; Thiobencarb; Tiocarbazil; Tralkoxydim; triallate; Triasulfuron; Triaziflam; Tribenuron; Tribenuron-methyl; Tribenuron-methyl; trichloroacetic acid; Triclopyr; Triclopyr-butotyl; Triclopyr-triethylammonium; Trietazine; Trifluralin; Triflusulfuron; Triflusulfuron-methyl; Vernolate: YRC 2388; dichloropicolinic acid, aryloxyalkanoic acids; fluoroxypyr; MCPP; fluoroglycofen; halosafen; di-allate; terbutryne; aminotriazole; sulphosate; tridiphane; propoxycarbazone-sodium; 4-amino-n-(1,1-dimethylethyl)-4,5-dihydro-3-(1-methylethyl)-5-oxo-1H-1,2,4-triazole-1-carboxamide; and benzoic acid 2-((((4,5-dihdydro-4-methyl-5-oxo-3-propoxy-1H-1,2,4-triazol-1-yl)carbonyl)amino)sulfonyl) methyl ester.

Examples of suitable agricultural active ingredients also include, but are not limited to, bronopol, dichlorophen, nitrapyrin, octhilinone, furancarboxylic acid, oxytetracyclin, probenazole, and tecloftalam.

Additionally, suitable examples of agricultural active ingredients include, but are not limited to, ammonium sulfate; ammonium salts (such as ammonium chloride, or ammonium phosphates); nitrates (such as ammonium nitrate, calcium nitrate, sodium nitrate, or potassium nitrate); urea; substituted ureas (such as urea-aldehyde condensates or methylene ureas); inorganic phosphates (such as ammonium phosphate or potassium phosphate); potassium salts (such as potassium nitrate, potassium phosphate, potassium sulfate, or potassium chloride); and trace elements necessary for satisfactory crop growth (such as zinc, iron, copper, cobalt, molybdenum, and manganese). Trace elements may be present as their salts, or as anions such as molybdate or as a complex. For example, iron may be present as a complex with ethylenediamine tetraacetic acid.

Also, suitable examples of agricultural active ingredients include, but are not limited to, chlorcholine chloride and ethephon.

The agricultural active ingredient(s) selected will typically need to comport with a specific application need. Accordingly, the agricultural active ingredient(s) selected may be present in varying amounts, as well as in varying physical forms, such as solid particles, liquid, or semiliquid form. In some embodiments, the agricultural active ingredient(s) selected may be between 0 and 50% (by weight) of a composition described herein.

In some embodiments, Component (D) may be selected from Vitamin C, green tea extract, lidocaine, nicotine, niacinnamide, salicylic acid, ketoprofen, and ketoconazole.

In some embodiments, Component (D) may be selected from urea, ammonium nitrate, potassium nitrate, sodium nitrate, potassium phosphate, and ammonium phosphate.

### Optional Component (E), Surfactant

Generally, optional Component (E) comprises at least one surfactant selected from cationic, anionic, and/or nonionic surfactants, wherein the surfactant can be aqueous, non-aqueous, and/or in diluted or undiluted form.

Examples of suitable cationic surfactants include, but are not limited to, quaternary ammonium hydroxides such as octyl trimethyl ammonium hydroxide, dodecyl trimethyl ammonium hydroxide, hexadecyl trimethyl ammonium hydroxide, octyl dimethyl benzyl ammonium hydroxide, decyl dimethyl benzyl ammonium hydroxide, didodecyl dimethyl ammonium hydroxide, dioctadecyl dimethyl ammonium hydroxide, tallow trimethyl ammonium hydroxide and coco trimethyl ammonium hydroxide as well as corresponding salts of these materials, fatty amines and fatty acid amides and their derivatives, basic pyridinium compounds, and quaternary ammonium bases of benzimidazolines and poly(ethoxylated/propoxylated) amines.

Examples of suitable anionic surfactants include, but are not limited to, alkyl sulphates such as lauryl sulphate, polymers such as acrylates/C₁₀₋₃₀ alkyl acrylate crosspolymer alkylbenzenesulfonic acids and salts such as hexylbenzenesulfonic acid, octylbenzenesulfonic acid, decylbenzenesulfonic acid, dodecylbenzenesulfonic acid, cetylbenzenesulfonic acid and myristylbenzenesulfonic acid; the sulphate esters of monoalkyl polyoxyethylene ethers; alkylnapthylsulfonic acid; alkali metal sulfoccinates, sulfonated glyceryl esters of fatty acids such as sulfonated monoglycerides of coconut oil acids, salts of sulfonated monovalent alcohol esters, amides of amino sulfonic acids, sulfonated products of fatty acid nitriles, sulfonated aromatic hydrocarbons, condensation products of naphthalene sulfonic acids with formaldehyde, sodium octahydroanthracene sulfonate, alkali metal alkyl sulphates, ester sulphates, and alkarylsulfonates. Anionic surfactants include alkali metal soaps of higher fatty acids, alkylaryl sulfonates such as sodium dodecyl benzene sulfonate, long chain fatty alcohol sulfates, olefin sulfates and olefin sulfonates, sulfated monoglycerides, sulfated esters, sulfonated ethoxylated alcohols, sulfosuccinates, alkane sulfonates, phosphate esters, alkyl isethionates, alkyl taurates, and alkyl sarcosinates.

Examples of suitable non-ionic surfactants include, but are not limited to, condensates of ethylene oxide with long chain fatty alcohols or fatty acids such as a C12-C16 alcohol, condensates of ethylene oxide with an amine or an amide, condensation products of ethylene and propylene oxide, esters of glycerol, sucrose, sorbitol, fatty acid alkylol amides, sucrose esters, fluoro-surfactants, fatty amine oxides, polyoxyalkylene alkyl ethers such as polyethylene glycol long chain alkyl ether, polyoxyalkylene sorbitan ethers, polyoxyalkylene alkoxylate esters, polyoxyalkylene alkylphenol ethers, ethylene glycol propylene glycol copolymers and alkylpolysaccharides, polymeric surfactants such as polyvinyl alcohol (PVA) and polyvinylmethylether. In certain embodiments, the surfactant is a polyoxyethylene fatty alcohol or mixture of polyoxyethylene fatty alcohols. In other embodiments, the surfactant is an aqueous dispersion of a polyoxyethylene fatty alcohol or mixture of polyoxyethylene fatty alcohols.

In some embodiments, Component (E) may be selected from Tergitol™ 15-s-3, Tergitol™ 15-s-40, sorbitan monooleate, polylycol-modified trimethsilylated silicate, polyglycol-modified siloxanes, polyglycol-modified silicas, ethoxylated quaternary ammonium salt solutions, and cetyltrimethylammonium chloride solutions.

### Optional Component (F), Free-Radical Polymerizable Organopolysiloxanes

Generally, optional Component (F) comprises at least one free-radical polymerizable organopolysiloxane that is capable of undergoing free radical-catalyzed addition polymerization, and in some embodiments, can also undergo co-polymerization and/or cross-linking.

In some embodiments, such organopolysiloxanes have free radical polymerizable groups and can be polymeric or a mixture of oligomers and polymers, wherein polymeric organopolysiloxanes can either be homopolymeric or heteropolymeric. Suitable organopolysiloxanes can be linear, branched, hyperbranched or resinous in structure.

In some embodiments, Component (F) comprises organopolysiloxanes having at least one free radical polymerizable moiety per molecule, wherein such moieties are monofunctional, multifunctional, or a combination thereof. Thus, Component (F) can be a mixture of organopolysiloxanes differing in their degree of functionality and/or the nature of the free radical polymerizable moieties. The organopolysiloxanes of Component (F) can also vary in consistency from a fluid to a gum. For example, the organopolysiloxane can be a fluid, a solid, or a solid that becomes flowable at an elevated temperature or by the application of shear. In some embodiments, the organopolysiloxanes have a viscosity of from about 1 cP to about 5,000,000 cP at 25° C.; alternatively, from about 50 cP to about 500,000 cP at 25° C.; alternatively, from about 100 cP to about 100,000 cP at 25° C.

The organopolysiloxanes of Component (F) may also have a glass transition temperature or, upon polymerization or crosslinking, form particles that have a glass transition temperature, wherein the resulting silicone composition undergoes marked changes in its viscosity under the temperatures of use. Such compositions are particularly useful for encapsulation of active ingredients that are released by the introduction of heat.

In some embodiments, Component (F) may comprise free radical polymerizable organopolysiloxanes having formulae selected from:

R¹₃SiO(R¹₂SiO)ₐ(R¹R²SiO)_{b}SiR¹₃; (1)

wherein a has a value of zero to 20,000 and b has a value of 1 to 20,000; and wherein each R¹ group is independently a hydrogen, halogen, or a monovalent organic group, and each R² group is independently a monovalent unsaturated organic group; and

R³₂R⁴SiO(R³₂SiO)_{c}(R³R⁴SiO)_{d}SiR³₂R⁴; (2)

wherein c has a value of zero to 20,000, and d has a value of zero to 20,000; and wherein each R³ is independently a hydrogen, halogen, or a monovalent organic group, and each R⁴ group is independently a monovalent unsaturated organic group.

Suitable R¹ and R³ groups include, but are not limited to, hydrogen; organic groups (linear and/or branched) such as alkyl groups, haloalkyl groups, alkenyl groups, alkynyl groups, acrylate functional groups, and methacrylate functional groups; and other organic functional groups such as glycidyl groups, amine groups, ether groups, cyanate ester groups, isocyano groups, ester groups, carboxylic acid groups, carboxylate salt groups, succinate groups, anhydride groups, mercapto groups, sulfide groups, azide groups, phosphonate groups, phosphine groups, masked isocyano groups, and hydroxyl groups. Examples of such groups include, but are not limited to, acrylate functional groups such as acryloyloxypropyl groups and methacryloyloxypropyl groups; alkyl groups such as methyl, ethyl, propyl, isopropyl, n-butyl, s-butyl, and t-butyl groups; alkenyl groups such as vinyl, allyl, and butenyl groups; alkynyl groups such as ethynyl and propynyl groups; aromatic groups such as phenyl, tolyl, and xylyl groups; cyanoalkyl groups such as cyanoethyl and cyanopropyl groups; halogenated hydrocarbon groups such as 3,3,3-trifluoropropyl, 3-chloropropyl, dichlorophenyl, and 6,6,6,5,5,4,4,3,3-nonafluorohexyl groups; alkenyloxypoly(oxyalkyene) groups such as allyloxy(polyoxyethylene), alkyloxypoly(oxypropylene), and allyloxy-poly(oxypropylene)-co-poly(oxyethylene) groups; alkyloxypoly(oxyalkyene) groups such as propyloxy(polyoxyethylene), propyloxypoly(oxypropylene), and propyloxy-poly(oxypropylene)-co-poly(oxyethylene) groups; halogen substituted alkyloxypoly(oxyalkyene) groups such as perfluoropropyloxy(polyoxyethylene), perfluoropropyloxypoly(oxypropylene), and perfluoropropyloxy-poly(oxypropylene)-co-poly(oxyethylene) groups; alkoxy groups such as methoxy, ethoxy, n-propoxy, isopropoxy, n-butoxy, and ethylhexyloxy groups; aminoalkyl groups such as 3-aminopropyl, 6-aminohexyl, 1 1-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole groups; epoxyalkyl groups such as 3-glycidoxypropyl, 2-(3,4,-epoxycyclohexyl)ethyl, and 5,6-epoxyhexyl groups; ester functional groups such as actetoxyethyl and benzoyloxypropyl groups; hydroxy functional groups such as hydroxy and 2-hydroxyethyl groups; isocyanate and masked isocyanate functional groups such as 3-isocyanatopropyl, tris-3-propylisocyanurate, propyl-t-butylcarbamate, and propylethylcarbamate groups; aldehyde functional groups such as undecanal and butyraldehyde groups; anhydride functional groups such as 3-propyl succinic anhydride and 3-propyl maleic anhydride groups; carboxylic acid functional groups such as 3-carboxypropyl and 2-carboxyethyl groups; and metal salts of carboxylic acids such as the zinc, sodium, or potassium salts of 3-carboxypropyl and 2-carboxyethyl.

Suitable R² and R⁴ groups include, but are not limited to, monovalent alkenyl and alkynyl groups having 2-12 carbon atoms groups such as vinyl, allyl, butenyl, ethynyl, and propynyl groups; alkenyloxypoly(oxyalkyene) groups such as allyloxy(polyoxyethylene), allyloxypoly(oxypropylene), and allyloxy-poly(oxypropylene)-co-poly(oxyethylene) groups; acrylate functional groups such as acryloyloxypropyl and methacryloyloxypropyl groups; and halogen-substituted analogs thereof. In certain embodiments, R² and R⁴ are selected from acrylate groups and methacrylate groups.

Some representative examples of Component (F) include, but are not limited to, methacryloxypropyldimethylsiloxy-terminated polydimethylsiloxane; acryloxypropyldimethylsiloxy-terminated polydimethylsiloxane, 1,3-bis(methacryloxypropyl)tetramethyldisiloxane, 1,3-bis(acryloxypropyl)tetramethyldisiloxane, 1,3-bis(methacryloxymethyl)tetramethyldisiloxane, 1,3-bis(acryloxymethyl)tetramethyldisiloxane, α,ω,-methacryloxymethyldimethylsilyl terminated polydimethylsiloxane, methacryloxypropyl-terminated polydimethylsiloxane, α,ω-acryloxymethyldimethylsilyl terminated polydimethylsiloxane, methacryloxypropyldimethylsilyl terminated polydimethylsiloxane, α,ω-acryloxypropyldimethylsilyl terminated polydimethylsiloxane, pendant acrylate and methacrylate functional polymers such as poly(acryloxypropyl-methylsiloxy) polydimethylsiloxane and poly(methacryloxypropyl-methylsiloxy) polydimethylsiloxane copolymers, telechelic polydimethylsiloxanes having multiple acrylate or methacrylate functional groups including those formed via a Michael addition reaction of multi-acrylate or multi-methacrylate monomers to amine-terminated polydimethylsiloxanes, and combinations thereof. Also suitable for use as free radical polymerizable organosilicon compounds include monofunctional acrylate or methacrylate terminated organopolysiloxanes such as polydimethylsiloxane terminated at one end by a methacryloxypropyldimethylsilyl group and terminated at the other end by n-butyldimethylsilyl groups.

In some embodiments, Component (F) may comprise a siloxane resin selected from MQ resins having R⁵₃SiO_{1/2} units and SiO_{4/2} units; TD resins having R⁵SiO_{3/2} units and R⁵₂SiO_{2/2} units; MT resins having R⁵₃SiO_{1/2} units and R⁵SiO_{3/2} units; MTD resins having R⁵₃SiO_{1/2} units, R⁵SiO_{3/2} units, and R⁵₂SiO_{2/2} units, and combinations thereof; wherein each R⁵ group is independently a monovalent organic group having from 1-20 carbon atoms. In some embodiments, R⁵ has from 1-10 carbon atoms. In some embodiments, at least one R⁵ group is a free radical polymerizable unsaturated organic group.

Suitable examples of R⁵ include, but are not limited to, acrylate functional groups such as acryloxyalkyl groups; methacrylate functional groups such as methacryloxyalkyl groups; cyanofunctional groups; monovalent hydrocarbon groups; and combinations thereof. The monovalent hydrocarbon groups may include alkyl groups such as methyl, ethyl, propyl, isopropyl, n-butyl, s-butyl, t-butyl, pentyl, neopentyl, octyl, undecyl, and octadecyl groups; cycloalkyl groups such as cyclohexyl groups; alkenyl groups such as vinyl, allyl, butenyl, and hexenyl groups; alkynyl groups such as ethynyl, propynyl, and butynyl groups; aryl groups such as phenyl, tolyl, xylyl, benzyl, and 2-phenylethyl groups; halogenated hydrocarbon groups such as 3,3,3-trifluoropropyl, 3-chloropropyl, dichlorophenyl, and 6,6,6,5,5,4,4,3,3-nonafluorohexyl groups; and combinations thereof. The cyano-functional groups may include cyanoalkyl groups such as cyanoethyl and cyanopropyl groups, and combinations thereof.

R⁵ may also include alkyloxypoly(oxyalkyene) groups such as propyloxy(polyoxyethylene), propyloxypoly(oxypropylene) and propyloxy-poly(oxypropylene)-co-poly(oxyethylene) groups, halogen substituted alkyloxypoly(oxyalkyene) groups such as perfluoropropyloxy(polyoxyethylene), perfluoropropyloxypoly(oxypropylene) and perfluoropropyloxy-poly(oxypropylene) copoly(oxyethylene) groups, alkenyloxypoly(oxyalkyene) groups such as allyloxypoly(oxyethylene), allyloxypoly(oxypropylene) and allyloxy-poly(oxypropylene) copoly(oxyethylene) groups, alkoxy groups such as methoxy, ethoxy, n-propoxy, isopropoxy, n-butoxy and ethylhexyloxy groups, aminoalkyl groups such as 3-aminopropyl, 6-aminohexyl, 11-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole groups, hindered aminoalkyl groups such as tetramethylpiperidinyl oxypropyl groups, epoxyalkyl groups such as 3-glycidoxypropyl, 2-(3,4,-epoxycyclohexyl)ethyl, and 5,6-epoxyhexyl groups, ester functional groups such as acetoxymethyl and benzoyloxypropyl groups, hydroxyl functional groups such as hydroxy and 2-hydroxyethyl groups, isocyanate and masked isocyanate functional groups such as 3-isocyanatopropyl, tris-3-propylisocyanurate, propyl-t-butylcarbamate, and propylethylcarbamate groups, aldehyde functional groups such as undecanal and butyraldehyde groups, anhydride functional groups such as 3-propyl succinic anhydride and 3-propyl maleic anhydride groups, carboxylic acid functional groups such as 3-carboxypropyl, 2-carboxyethyl, and 10-carboxydecyl groups, metal salts of carboxylic acids such as zinc, sodium, and potassium salts of 3-carboxypropyl and 2-carboxyethyl groups, and combinations thereof.

Some specific examples of suitable siloxane resins that can be used as Component (F) include, but are not limited to, M^{Methacryloxymethyl}Q resins; M^{Methacryloxypropyl}Q resins; MT^{Methacryloxymethyl}T resins; MT^{Methacryloxypropyl}T resins; MDT^{Methacryloxymethyl}T^{Phenyl}T resins; MDT^{Methacryloxypropyl}T^{Phenyl}T resins; M^{Vinyl}T^{Phenyl} resins; TT^{Methacryloxymethyl} resins; TT^{Methacryloxypropyl} resins; T^{Phenyl}T^{Methacryloxymethyl} resins; T^{Phenyl}T^{Methacryloxypropyl} resins; TT^{Phenyl}T^{Methacryloxymethyl} resins; TT^{Phenyl}T^{Methacryloxypropyl} resins; and combinations thereof.

Siloxane resins may be prepared by any method known in the art. In some embodiments, the resin is made by treating a resin copolymer produced by a silica hydrosol capping process with an alkenyl including endblocking reagent. This preferably includes reacting a silica hydrosol under acidic conditions with a hydrolyzable triorganosilane such as trimethylchlorosilane, a siloxane such as hexamethyldisiloxane, and combinations thereof, and then recovering a copolymer having M and Q groups including 2 to 5% wt of hydroxyl groups. The copolymer may be reacted with an endblocking agent including unsaturated organic groups and an endblocking agent free of aliphatic unsaturation in amounts sufficient to provide 3 to 30 mole percent of unsaturated organofunctional M, D or T groups in the resin relative to the sum of all M, D, T and Q units comprising the resin. Suitable endblocking agents include silazanes, siloxanes, silanes, and combinations thereof.

In some embodiments, Component (F) may be selected from methacrylate-functional polydimethylsiloxanes and resins, and acrylate-functional polydimethylsiloxanes and resins.

### Optional Component (G), Organoborane Free Radical Initiator

Generally, optional Component (G) comprises at least one organoborane compound that is capable of generating a free radical and initiating free radical addition polymerization and/or crosslinking Stabilized organoborane compounds that render the organoborane non-pyrophoric at ambient conditions may be used. In some embodiments, Component (G) is a complex formed between an organoborane and a suitable organonitrogen (for example, an amine) that renders the complex stable at ambient conditions, wherein a free radical is generated (and polymerization is initialized) upon introduction of an organonitrogen-reactive compound in the presence of oxygen. In some embodiments, Component (G) is an organoborane compound wherein a free radical is generated (and polymerization is initiated) upon heating. In some embodiments, Component (G) is a solvent-stabilized organoborane (for example, a solution of a trialkylborane in THF) where the solvent is allowed to evaporate to liberate the borane and thereby create a radical.

In some embodiments, Component (G) is an organoborane-organonitrogen complex that may be selected from complexes having the formula:

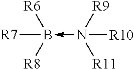

wherein B represents boron and N represents nitrogen; wherein at least one of R6, R7, and R8 contains one or more silicon atoms with the silicon-containing group(s) covalently attached to boron; wherein R6, R7, and R8 are groups that can be independently selected from hydrogen, a cycloalkyl group, a linear or branched alkyl group having 1-12 carbon atoms on the backbone, an alkylaryl group, an organosilane group such as an alkylsilane or an arylsilane group, an organosiloxane group, an alkylene group capable of functioning as a covalent bridge to another boron atom, a divalent organosiloxane group capable of function as a covalent bridge to another boron atom, or halogen substituted homologues thereof; wherein R9, R10, and R11 are groups that yield an amine compound or a polyamine compound capable of complexing with boron and are independently selected from hydrogen, an alkyl group containing 1-10 carbon atoms, a halogen substituted alkyl group containing 1-10 carbon atoms, or an organosilicon functional group; and wherein at least two of the R6, R7, and R8 groups and at least two of the R9, R10, and R11 groups can combine to form heterocyclic structures, provided that the sum of the number of atoms from the two combining groups does not exceed 11.

In some embodiments, Component (G) may be selected from alkylborane-organonitrogen complexes that include, but are not limited to, trialkylborane-organonitrogen complexes comprising trialkylboranes having the formula BR″₃, wherein R″ represents linear and branched aliphatic or aromatic hydrocarbon groups containing 1-20 carbon atoms. Examples of suitable trialkylboranes include, but are not limited to, trimethylborane, tri-n-butylborane, tri-n-octylborane, tri-sec-butylborane, tridodecylborane, and phenyldiethylborane.

Examples of suitable organonitrogens for forming the organoborane-organonitrogen complexes of Component (G) include, but are not limited to, 1,3 propane diamine; 1,6-hexanediamine; methoxypropylamine; pyridine; isophorone diamine; and silicon-containing amines such as 3-aminopropyltrimethoxysilane, 3-aminopropyltriethoxysilane, 2-(trimethoxysilylethyl)pyridine, aminopropylsilanetriol, 3-(m-aminophenoxy)propyltrimethoxysilane, 3-aminopropyldiisopropylmethoxysilane, aminophenyltrimethoxysilane, 3-aminopropyltris(methoxyethoxethoxy)silane, N-(2-aminoethyl)-3-aminopropyltrimethoxysilane, N-(6-aminohexyl)aminomethyltrimethoxysilane, N-(2-aminoethyl)-h I-aminoundecyltrimethoxysilane, (aminoethylaminomethyl)pbenethyltrimethoxysilane, N-(2-amino ethyl)-3-aminopropylmethyldimethoxysilane, N-(2-aminoethyl)-3-aminoisobutylmethyldimethoxysilane, and (3-trimethoxysilylpropyl)diethylene-triamine.

In some embodiments, nitrogen-containing compounds that may be useful for forming the organoborane-organonitrogen complexes of Component (G) may be selected from organopolysiloxanes having least one amine functional group. Examples of suitable amine functional groups include, but are not limited to, 3-aminopropyl, 6-aminohexyl, 11-aminoundecyl, 3-(N-allylamino)propyl, N-(2-aminoethyl)-3-aminopropyl, N-(2-aminoethyl)-1-aminoisobutyl, p-aminophenyl, 2-ethylpyridine, and 3-propylpyrrole. Such organopolysiloxanes include, but are not limited to, those having formulas similar to the previously described formulas (1) and (2). Other nitrogen-containing compounds that may be useful for forming the organoborane-organonitrogen complexes of Component (G) include, but are not limited to, N-(3-triethyoxysilylpropyl)-4,5-dihydroimidazole, ureidopropyltriethoxysilane, siloxanes having formulas similar to the previously described formulas (1) and (2), and organopolysiloxane resins in which at least one group is an imidazole, amidine, or ureido functional group.

In some embodiments, a free radical is generated by, and polymerization and/or crosslinking is initiated by, heating an organoborane compound (preferably organoborane-organonitrogen complex) or by simply exposing an anaerobically contained alkylborane of Component (G) to air. In some embodiments, a free radical is generated by, and polymerization and/or crosslinking is initiated by, heating an organoborane-organonitrogen complex of Component (G), wherein heating causes dissociation of the complex. In some embodiments, a free radical is generated by, and polymerization and/or crosslinking is initiated by, combining an organonitrogen-reactive compound with an organoborane-organonitrogen complex of Component (G) in an oxygen environment, wherein the combination causes dissociation of the complex. With respect to the latter, a free radical can be generated at temperatures below the dissociation temperature of the organoborane-organonitrogen complex, such as at or below ambient temperature.

Although organonitrogen-stabilized organoborane compounds are particularly useful as Component (G), one of skill in the art will understand that any organoborane may be used. Examples of alternate stabilized forms of organoboranes useful for this invention, include ring stabilized compounds, such as 9-BBN, or solvent complexed organoboranes such as trialkylborane-THF solutions.

In some embodiments, Component (G) is a trialkylborane-organonitrogen complex wherein the trialkylborane is selected from triethylborane, tri-n-butylborane, tri-n-octylborane, tri-sec-butylborane, and tridodecylborane. In some embodiments, Component (G) may be selected from triethylborane-propanediamine (TEB-PDA), triethylborane-butylimidazole (TEB-BI), and triethylborane-methoxypropylamine (TEB-MOPA) complexes, and tri-n-butyl methoxypropyl amine complexes.

### Optional Organonitrogen-Reactive Compound

In some embodiments, if an organoborane-organonitrogen complex of optional Component (G) is used, at least one organonitrogen-reactive compound is also used. When combined with Component (G) and exposed to an oxygenated environment such as ambient air, the at least one organonitrogen-reactive compound is capable of causing the organoborane-organonitrogen complex to dissociate, thereby initiating free radical polymerization and/or crosslinking. The presence of such an organonitrogen-reactive compound allows for polymerization and/or crosslinking to occur rapidly at temperatures below the dissociation temperature of the organoborane-organonitrogen complexes of Component (G), including at room temperature and below.

Some examples of suitable organonitrogen-reactive compounds include, but are not limited to, mineral acids, Lewis acids, carboxylic acids, carboxylic acid derivatives such as anhydrides and succinates, carboxylic acid metal salts, isocyanates, aldehydes, epoxides, acid chlorides, and sulphonyl chlorides, acetic acid, acrylic acid, methacrylic acid, polyacrylic acid, polymethacrylic acid, methacrylic anhydride, undecylenic acid, oleic acid, citric acid, stearic acid, levulinic acid, 2-carboxyethyl acrylate, isophorone diisocyanate monomers or oligomers, methacryloylisocyanate, 2-(methacryloyloxy)ethyl acetoacetate, undecylenic aldehyde, and dodecyl succinic anhydride.

Additionally, organosilanes or organopolysiloxanes having organonitrogen-reactive groups can be suitable organonitrogen-reactive compounds. Such compounds include, but are not limited to, 3-isocyanatopropyltrimethoxysilane; 3-glycidoxypropyltrimethoxysilane; propylsuccinic anhydride functionalized linear, branched, resinous, and hyperbranched organopolysiloxanes; cyclohexenyl anhydride functional linear, resinous, and hyperbranched organopolysiloxanes; carboxylic acid functionalized linear, branched, resinous, and hyperbranched organopolysiloxanes such as carboxydecyl terminated oligomeric or polymeric polydimethylsiloxanes; and aldehyde functionalized linear, branched, resinous, and hyperbranched organopolysiloxanes such as undecylenic aldehyde-terminated oligomeric or polymeric polydimethylsiloxanes.

Other suitable organonitrogen-reactive compounds are silicon containing compounds that, when exposed to moisture, release an acid that causes the organoborane-organonitrogen complex of Component (G) to disassociate. Such compounds include, but are not limited to, halo silanes, acid anhydride (carboxylic acid) siloxanes, acetoxy siloxanes (such as ethylriacetoxysiloxane and methyl triacetoxysiloxane), alkyl silicic acids, esters of carboxylic acids and silanols, acid chloride siloxanes.

Further examples of compounds that can be useful as organonitrogen-reactive compounds are those capable of generating organonitrogen-reactive groups when exposed to ultraviolet radiation, such as iodonium salts containing [SbF₆ ]⁻ counterions. With such compounds, it may be useful to also include a photosensitizing compound such as isopropylthioxanthone.

One of skill in the art will recognize that the selection of the organonitrogen-reactive compound will depend upon, among other things, the nature of Component (G).

When an organonitrogen-reactive compound is used, free radical generation requires the presence of oxygen. In some embodiments, merely exposing the organonitrogen-reactive compound or the composition containing the organonitrogen-reactive compound to air is sufficient to induce polymerization. In some embodiments, the oxygen dissolved in one or more of the other components of the composition is sufficient. To prevent premature polymerization in the presence of oxygen, Component (G) and the organonitrogen-reactive compound may be physically or chemically isolated until just prior to the desired time to initiate polymerization and/or crosslinking reactions. For example, the composition may be prepared initially as two separate components that are combined into one, just prior to the initiation of polymerization and/or crosslinking. The remaining components of the composition may be distributed in any manner between the two components.

In some embodiments, when an organoborane-organonitrogen complex of optional Component (G) is used, an organonitrogen-reactive compound is not required. In such cases, free radical polymerization may be initiated by exposing the organoborane compound to air, by thermal activation, or via radiation. In the case of thermal activation, the temperature to which the one or more components of the composition must be heated to initiate polymerization is dictated by the nature of the organoborane compound selected as Component (G). For example, if an organoborane-organonitrogen complex is selected as Component (G), the binding energy of the complex will dictate the temperature to which the composition must be heated to initiate dissociation of the complex and polymerization. In some embodiments, Component (G) may be heated prior to its introduction with the other components of the composition. In other embodiments, Component (G) and at least one other component are heated prior to the introduction of any remaining components of the composition.

### Additional Optional Components

The provided compositions may optionally include additional components. Without limitation, examples of such optional additional components include surfactants; emulsifiers; dispersants; rheology modifiers such as thickeners; density modifiers; aziridine stabilizers; polymers; diluents; acid acceptors; antioxidants; heat stabilizers; flame retardants; scavenging agents; silylating agents; foam stabilizers; solvents; diluents; plasticizers; fillers and inorganic particles, pigments, dyes and dessicants.

Provided compositions may contain a number of optional components selected from those known in the state of the art to be ingredients in personal and healthcare formulations. Illustrative, non-limiting examples include surfactants, solvents, powders, coloring agents, thickeners, waxes, stabilizing agents, pH regulators, and silicones. Thickening agents may optionally be added to the aqueous phase of the compositions to provide a convenient viscosity. For example, viscosities within the range of 500 to 25,000 mm²/s at 25° C. or more, alternatively in the range of 3,000 to 7,000 mm²/s at 25° C., are usually suitable. Suitable thickening agents are exemplified by sodium alginate; gum arabic; polyoxyethylene; guar gum; hydroxypropyl guar gum; ethoxylated alcohols, such as laureth-4 or polyethylene glycol 400; cellulose derivatives exemplified by methylcellulose, methylhydroxypropylcellulose, hydroxypropylcellulose, polypropylhydroxyethylcellulose; starch and starch derivatives exemplified by hydroxyethylamylose and starch amylose; locust bean gum; electrolytes exemplified by sodium chloride and ammonium chloride; saccharides such as fructose and glucose; and derivatives of saccharides such as PEG-120, methyl glucose diolate; or mixtures of two or more of these. Alternatively the thickening agent is selected from cellulose derivatives, saccharide derivatives, and electrolytes, or from a combination of two or more of the above thickening agents exemplified by a combination of a cellulose derivative and any electrolyte, and a starch derivative and any electrolyte. The thickening agent may be present in shampoo compositions of the present invention in an amount sufficient to provide a viscosity in the final shampoo composition of from 500 to 25,000 mm²/s. The thickening agent may be present in an amount from about 0.05 to 10 wt %; alternatively from about 0.05 to 5 wt %, based on the total weight of the composition. Thickeners based on acrylate derivatives, such as polyacrylate crosspolymer, Acrylates/C1030 Alkyl Acrylate crosspolymer, polyacrylamide derivatives, sodium polyacrylate may also be added.

Stabilizing agents may optionally be used in the water phase of the provided compositions. Suitable water phase stabilizing agents can include alone or in combination one or more electrolytes, polyols, alcohols such as ethyl alcohol, and hydrocolloids. Typical electrolytes are alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate. When the stabilizing agent is, or includes, an electrolyte, it amounts to about 0.1 to 5 wt % and more alternatively 0.5 to 3 wt % of the total composition. The hydrocolloids include gums, such as Xantham gum or Veegum and thickening agents, such as carboxymethyl cellulose. Polyols, such as glycerine, glycols, and sorbitols can also be used. Alternative polyols are glycerine, propylene glycol, sorbitol and butylene glycol. If a large amount of a polyol is used, one need not add the electrolyte. However, it is typical to use a combination of an electrolyte, a polyol and a hydrocolloid to stabilize the water phase, e.g. magnesium sulfate, butylene glycol and Xantham gum.

Other optional components can include powders and pigments. A powder composition can be generally defined as dry, particulate matter having a particle size of 0.02-50 microns. The particulate matter may be colored or non-colored (for example white). Suitable powders include, but are not limited to, bismuth oxychloride, titanated mica, fumed silica, spherical silica beads, polymethylmethacrylate beads, boron nitride, aluminum silicate, aluminum starch octenylsuccinate, bentonite, kaolin, magnesium aluminum silicate, silica, silica silylate, talc, mica, titanium dioxide, nylon, silk powder. The above-mentioned powders may be surface treated to render the particles hydrophobic in nature. The powder component also comprises various organic and inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Inorganic pigments generally consist of insoluble metallic salts of certified color additives, referred to as the Lakes or iron oxides. A pulverulent coloring agent, such as carbon black, chromium or iron oxides, ultramarines, manganese pyrophosphate, iron blue, and titanium dioxide, pearlescent agents, generally used as a mixture with colored pigments, or some organic dyes, generally used as a mixture with colored pigments and commonly used in the cosmetics industry, can be added to the composition. In general, these coloring agents can be present in an amount by weight from 0 to 20% with respect to the weight of the final composition.

Pulverulent inorganic or organic fillers can also be added, generally in an amount by weight from 0 to 40% with respect to the weight of the final composition. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked, copolymer microspheres such as EXPANCEL (Nobel Industrie), polytrap and silicone resin microbeads (TOSPEARL from Toshiba, for example).

Waxes or wax-like materials may be optional components of the provided compositions, wherein such components generally have a melting point range of 35 to 120° C. at atmospheric pressure. Waxes in this category include synthetic wax, ceresin, paraffin, ozokerite, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, soy waxes or mixtures thereof. Mention may be made, among the waxes capable of being used as non-silicone fatty substances, of animal waxes, such as beeswax; vegetable waxes, such as carnauba, candelilla wax, mineral waxes, for example paraffin or lignite wax or microcrystalline waxes or ozokerites; synthetic waxes, including polyethylene waxes, and waxes obtained by the Fischer-Tropsch synthesis. Mention may be made, among the silicone waxes, of polymethylsiloxane alkyls, alkoxys and/or esters.

Water soluble or water dispersible silicone polyether compositions may also be optional components. These are also known as polyalkylene oxide silicone copolymers, silicone poly(oxyalkylene) copolymers, silicone glycol copolymers, or silicone surfactants. These can be linear rake or graft type materials, ABA or ABn type where the B is the siloxane polymer block, and the A is the poly(oxyalkylene) group. The poly(oxyalkylene) group can consist of polyethylene oxide, polypropylene oxide, or mixed polyethylene oxide/polypropylene oxide groups. Other oxides, such as butylene oxide or phenylene oxide are also possible.

Compositions according to embodiments of the present invention can be used in o/w, s/w, w/o, w/s, and non-aqueous o/o, o/s, and s/o emulsions or multiple phase emulsions using silicone emulsifiers. Typically the water-in-silicone emulsifier in such formulation is non-ionic and is selected from polyoxyalkylene-substituted silicones (rake or ABn type), silicone alkanolamides, silicone esters and silicone glycosides. Suitable silicone-based surfactants are well known in the art, and have been described for example in U.S. Pat. No. 4,122,029 (Gee et al.), U.S. Pat. No. 5,387,417 (Rentsch), and U.S. Pat. No. 5,811,487 (Schulz et al), JP 2001-294512.

Water-soluble solvents may also be optional components in the hydrogel. Examples include acetonitrile, tetrahydrofuran, acetone, 1,4-dioxane, dimethylsulfoxide.

When a provided composition is an oil-in-water emulsion, it will include common ingredients generally used for preparing emulsions such as but not limited to non ionic surfactants well known in the art to prepare o/w emulsions. Examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, and polyoxyalkylene glycol modified polysiloxane surfactants.

A composition according to embodiments of the invention can also be under the form of aerosols in combination with propellant gases, such as carbon dioxide, nitrogen, nitrous oxide, volatile hydrocarbons such as butane, isobutane, or propane and chlorinated or fluorinated hydrocarbons such as dichlorodifluoromethane and dichlorotetrafluoroethane or dimethylether.

### Surface-Modified Hydrogels, Hydrogel Pastes, and Hydrogel Microparticles

In various embodiments, provided are hydrogels, hydrogel pastes, and hydrogel microparticles having siloxane coated surfaces. Such hydrogels and hydrogel pastes are prepared by a method comprising treating a hydrogel with Component (A), at least one amino-functional organosilicon compound, to form at least one siloxane-coated surface on the hydrogel; wherein the hydrogel comprises (i) Component (B), at least one organic polymer comprising amine-reactive groups selected from carboxy-functional groups, sulfonic acid-functional groups, epoxy groups, or combinations thereof, the polymer being compatible with water, water-compatible alcohols, or combinations thereof; and (ii) Component (C), at least one absorbable solvent selected from water, water-compatible alcohols, and combinations thereof.

Hydrogel microparticles are prepared by a method comprising treating hydrogel microparticles with Component (A), at least one amino-functional organosilicon compound, to form microparticles having at least one siloxane-coated surface; wherein the microparticles comprise Component (B), at least one organic polymer comprising amine-reactive groups selected from carboxy-functional groups, sulfonic acid-functional groups, epoxy groups, or combinations thereof, the polymer being compatible with water, water-compatible alcohols, or combinations thereof; and wherein treatment occurs in the presence of Component (C), at least one absorbable solvent selected from water, water-compatible alcohols, and combinations thereof.

In some embodiments, the siloxane-modified hydrogels and microparticles are prepared in the presence of Component (E).

In some embodiments, optional Components (F) and (G) are used to provide a secondary siloxane coating of the hydrogel. In some embodiments, optional Components (F), (G), and an organonitrogen-reactive compound are used to provide a secondary siloxane coating of the hydrogel. In some embodiments, optional Components (F) and (G), or (F), (G), and an organonitrogen-reactive compound, are added after Components (A) and (B) are allowed to mix. In further embodiments involving optional Components (F) and (G), the ratio of Component (A) to Component (B) is kept at a sufficiently low level to leave an excess of amine-reactive groups in Component (B) that are then able to serve in lieu of the optional organonitrogen-reactive compound.

In some embodiments, the amount of Component (A) used is dependent, in part, upon the nature and amount of Component (B) present in the hydrogel or microparticle, as well as the surface area of the hydrogel or microparticle, and thickness of coating desired. While good results have been obtained with a weight ratio of Component (A)/Component (B) of from about 0.5 to about 5, one of skill in the art will appreciate that the ratios described herein are not limiting.

In some embodiments, the untreated hydrogel or hydrogel microparticles comprise or are treated with Component (C). In some embodiments, Component (C) may be introduced (for example, by addition, exposure, contact, mixing, or combinations thereof) into the hydrogel prior to introduction of Component (B). In some embodiments, Component (C) may be introduced into the hydrogel after introduction of Component (B). In some embodiments, hydrogel microparticles may be treated with Component (C) before, after, or simultaneously with treatment with Component (A).

In some embodiments, the hydrogel or hydrogel microparticle (whether untreated, surface-modified, or both) optionally comprises or is optionally treated in the presence of Component (D). In some embodiments, the amount of Component (D) used is dependent, in part, upon its nature, its intended application, and amount needed to be beneficial. For example, in a personal or healthcare application, the amount of Component (D) used would be dependent, in part, upon the amount needed for beneficial delivery to the user.

In some embodiments, the amount of Component (F) used is dependent, in part, upon the nature and amount of Component (B) present in the hydrogel or microparticle, as well as the surface area of hydrogel or microparticle, and thickness of coating desired. While good results have been obtained with a weight ratio of Component (F)/Component (B) of from about 0.5 to about 5, one of skill in the art will appreciate that the ratios described herein are not limiting.

In some embodiments, the amount of Component (G) used is dependent upon a variety of factors. For example, the nature of Component (F), the water content of the gel or microparticle, the nature of the solvent present, the presence of a surfactant, and combinations thereof may be variables that affect the amount of Component (G) used in the provided methods. While good results have been obtained with a weight ratio of Component (G)/Component (F) of from about 0.01 to about 0.1, one of skill in the art will appreciate that the ratios described herein are not limiting.

In various embodiments, provided are surface-modified hydrogels and hydrogel microparticles prepared by the methods described herein. In some embodiments, such hydrogels and hydrogel microparticles are prepared at temperatures from about 5° C. to about 95° C. Thus, temperature may be from about 5° C. to about 10° C., from about 10° C. to about 15° C., from about 15° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., from about 70° C. to about 75° C., from about 75° C. to about 80° C., from about 80° C. to about 85° C., from about 85° C. to about 90° C., and from about 90° C. to about 95° C. In some embodiments, hydrogels and hydrogel microparticles may be prepared at temperatures from about 10° C. to about 35° C. Thus, the temperature may be 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., and 35° C.

In some embodiments, a siloxane polymer coating that resists solvent washing is formed on the surface of a hydrogel (for example, a gel slab or gel monolith) by the methods provided herein. The coating is formed on at least one surface and can serve as a barrier to the migration of water and/or alcohol into or from the hydrogel. The coating may also have time-release or delayed-release properties. In some embodiments, hydrogels having at least one hydrophobic surface are prepared according the methods provided herein. In some embodiments, hydrogels having at least one alcohol-resistant surface are prepared according to the methods provided herein.

In some embodiments, a siloxane polymer coating that resists solvent washing is formed on swollen hydrogel microparticles by the methods provided herein. The coating is formed on the surfaces of the microparticles and can serve as a barrier to the migration of water and/or alcohol into the hydrogel microparticles. The coating may also have time-release or delayed-release properties. In some embodiments, hydrogel microparticles having hydrophobic surfaces are prepared according the methods provided herein. In some embodiments, hydrogel microparticles having alcohol-resistant surfaces are prepared according to the methods provided herein.

In some embodiments, whether the resulting hydrogels or microparticles are solvent-compatible (dispersible) or solvent-resistant (i.e. permanently so) depends on the extent and nature of the coating that is formed. For example, coating thickness and cross-link density will be among the factors determining coating characteristics. In some embodiments, the coating formed on the surfaces of the hydrogels or microparticles can serve as a temporary barrier to the migration of solvent. In some embodiments, the coating formed on the surfaces of the hydrogels or microparticles can serve as a permanent barrier to the migration of solvent.

In some embodiments, a siloxane polymer coating is formed on the surface of a siloxane-coated hydrogel or hydrogel microparticle by the methods provided herein to create a thicker and more impervious hydrophobic shell around the hydrogel or hydrogel microparticle. For example, a double-encapsulated hydrogel microparticle may be formed by the methods provided herein.

### Pastes and Powders

Surface-modified hydrogels prepared by the provided methods may be dried and pulverized to form a powder. Such powders can be used in agricultural products or personal care and healthcare products, and since the powders can be formed from hydrogel compositions comprising active ingredients, the powders are ideal for delivering active ingredients in such products.

Surface-modified hydrogels and microparticles prepared by the provided methods may also be used to form pastes. Typically, a paste can be formed by applying high shear, cutting, abrasion, or impact to the hydrogel either as it is being formed or after it is formed, preferably in the presence of a non-reactive diluent. A paste can also be formed by dispersing microparticles in a non-reactive diluent.

The diluent chosen will, in some embodiments, depend upon the thickness of the siloxane coating formed on the hydrogel or microparticles. In some embodiments, a suitable diluent may be selected from water, water compatible alcohols, diols, polyols, and combinations thereof. Examples of suitable alcohols include, but are not limited to, methanol, ethanol, isopropyl alcohol, ethylene glycol, polyethylene glycol and combinations thereof. In some embodiments, a suitable diluent may be selected from water-immiscible silicones; organic compounds; and “ecologically-friendly” solvents, such as ionic liquids and supercritical fluids; and mixtures thereof. Examples of suitable diluents include, but are not limited to, linear, branched, hyperbranched and cyclic organosiloxane fluids, such as hexamethyldisiloxane, octamethyltrisiloxane, decamethyltetrasiloxane, and trimethylsilyl-terminated polydimethylsiloxane fluids having a viscosity of less than 1000 cP at 25° C., or a mixture thereof; capryllylmethyl trisiloxane; octamethylcyclotetrasiloxane; decamethylcyclopentasiloxane; and higher cyclosiloxanes and mixtures thereof. In some embodiments, trimethylsilyl-terminated polydimethylsiloxane fluids having a viscosity of from about 0.5 to about 100 cP at 25° C. are suitable diluents. Other suitable diluents include, but are not limited to, organic solvents immiscible with water, such as pentane, hexane, heptane, octane, cyclohexane, toluene, xylenes, ethyl acetate. Further examples of suitable diluents include, but are not limited to, organic oils such as isododecane, isohexadecane, isodecylneopentanoate, isononyl isononanoate, isoparaffin, isoalkane, and ionic liquids including, 1-ethenyl-3-ethyl-imidazolium hexafluorophosphate and tetrapropyl-ammonium tetracyanoborate, and supercriticial fluids such as supercritical carbon dioxide.

Any type of mixing and shearing equipment, such as a batch mixer, planetary mixer, single or multiple screw extruder, dynamic or static mixer, colloid mill, ball mill, homogenizer, sonolator, or a combination thereof, may be used to apply shear force to the hydrogel. The application of shear, cutting, abrasion, or impact may be performed any temperature, including sub-ambient conditions such as found in cryo-milling or cryo-fracturing, room temperature, or elevated temperature. Pastes made from the surface-modified hydrogel compositions and microparticles are stable and can have a wide range of viscosities, thereby making them particularly useful as bases for agricultural or personal care and healthcare products.

### EXAMPLES

The present invention will be better understood by reference to the following examples which are offered by way of illustration and which one of skill in the art will recognize are not meant to be limiting.

### Example 1

The following procedure was used to produce an siloxane-modified hydrogel paste throughout. The procedure was performed in ambient lab conditions.
1. The following ingredients were combined into an 8 oz. straight sided jar: 1.4 parts of a microparticulate crosslinked polyacrylic acid (μPAA-1) (Carbopol® ETD 2020 from Lubrizol Corp.), 1 part Sorbitan monooleate (Span 80 non-ionic surfactant), and 27.1 parts hexamethyldisiloxane (non-polar solvent). The mixture was stirred magnetically to achieve homogeneity.
2. 1.8 parts deionized water containing a water soluble food dye (Wilton Enterprises, Woodridge, Ill., “teal” icing colorant) was added drop wise and mixed with a magnetic stirrer. Stirring was continued for 10 minutes.
3. With mixing, 4.7 parts A-PDMS [poly(dimethyl, methyl (aminoethylaminoisobutyl) siloxane) polymer having a viscosity of approximately 130 cSt a 25° C. and 2 mol % methyl(aminoethylaminoisobutyl) siloxane units] were added drop wise and allowed to mix for an additional 15 minutes or until the composition thickened to the point of arresting the stir bar. The composition formed a viscous, thixotropic paste with the dye (as a model water-soluble active) uniformly distributed within the material.

### Example 2

The following procedure was used to produce hydrogel particles with a silicone coating. The process was carried out in ambient lab conditions.
1. 40 parts hexamethyldisiloxane were combined with 2 parts of a hydrophobically modified microparticulate crosslinked polyacrylic acid copolymer (μPAA-cp) [Carbopol® Ultrez 20 (Acrylates/C10-30 Alkyl Acrylate Cross-Polymer particles)] and 1 part Sorbitan monooleate in an 8 oz. straight sided jar. The mixture was stirred magnetically to achieve homogeneity.
2. With mixing 6 parts deionized water were added drop wise and allowed to mix for 10 minutes.
3. With mixing 4 parts A-PDMS were added drop wise and allowed to mix for an additional 15 minutes or until the composition thickened to the point of arresting the stir bar.
4. The resultant material was vacuum filtered through a 0.22 micron Teflon filter and rinsed with hexamethyldisiloxane and n-heptane.
5. The sample was dried in the vacuum oven at room temperature under vacuum (<5 mm Hg) for one hour.

The resulting material formed a gel-like material when mixed with deionized water. Evidence of hydrogel formation was confirmed by rheological testing, which showed a plateau modulus of 772 Pa. In the testing methodology, modulus measurements of various hydrogel samples at different hydration levels were obtained in frequency sweep mode using the parallel plate geometry on a Rheometrics Dynamic Analyzer RDA II rheometer. Modulus values reported are the dynamic storage modulus (G′) at 1% strain and a frequency of 10 rad/s in the plateau region of frequency sweeps. Sample thickness typically ranged between 1-3 mm. The samples were placed on the 40 mm diameter parallel plates and then trimmed to size using a Teflon spatula. Data gathered during the analysis were processed using TA Orchestrator Version V7.1.2.3. All tests were performed at room temperature.

Evidence of a silicone coating was confirmed by ATR-IR spectroscopy, which showed a distinct siloxane Si—O—Si peak at 1060 cm⁻¹ in the spectrum that is not present in the unmodified μPAA-cp sample. Spectroscopy methodology involved analyzing samples with a Nicolet 6700 FTIR equipped with a Smart Miracle single bounce attenuated total internal reflectance infrared (ATR-IR) attachment equipped with a ZnSe crystal. The samples were placed directly in contact with the crystal. Data were analyzed with Omnic 7.2 software.

### Example 3

2.7 parts of the sample prepared in Example 1 were added to 97.3 parts deionized water. It was observed that the silicone-modified hydrogel paste did not disperse into the water phase. It was further observed that the water soluble dye did not migrate to the water phase, confirming that the internally encapsulated water and water-soluble dye were isolated from the externally supplied water by the encapsulating silicone layer.

### Example 4

As a control, a 1% dispersion of an unmodified hydrogel was prepared by mixing μPAA-1 (as received) into deionized water at a concentration of 1 wt % solids. It was observed that the sample did not gel after a period of 2 days and remained as a low viscosity dispersion of Carbopol particles.

As another control, a drop of food coloring was added to 10.134 g of the prepared dispersion before neutralizing (thickening) the mixture with 2.047 g 0.1M KOH. Another mixture having 2.4% of this thickened mixture and 97.6% deionized water was prepared. It was observed that after 24 hours the dye had dispersed throughout the water phase. This further confirms that the silicone modification of Example 3 was successful in encapsulating the water soluble dye from the external water phase.

### Example 5

The following procedure was used to produce a siloxane modified hydrogel, substituting a cyclosiloxane solvent for hexamethyldisiloxane. The procedure was performed at ambient lab conditions.
1. The following ingredients were combined into an 8 oz. straight sided jar: 3.9 parts μPAA-1, 2.8 part Sorbitan monooleate, and 72.0 parts decamethylcyclopentasiloxane. The mixture was stirred magnetically to achieve homogeneity.
2. 9.5 parts water containing the teal water soluble dye were added drop wise with mixing. Stirring was continued for 10 minutes.
3. 11.7 parts A-PDMS were added drop wise and allowed to mix for an additional 15 minutes or until the mixture thickened sufficiently to arrest the stir bar. The final material formed a viscous, thixotropic paste with a uniform appearance similar to that of Example 1.

### Example 6

A sample was made using the method described for Example 1, but having the final composition of: 67.6% Hexamethyldisiloxane, 4.6% μPAA-1, 3.4% Sorbitan monooleate, 11.7% deionized water (with a water soluble dye), and 12.8% A-PDMS siloxane.

### Example 7

A cold blend of Example 6 was blended with an unmodified platinum cured silicone elastomer paste made following the procedure of Example 6 of U.S. Pat. No. 6,770,708 B2. It was mixed for 30 s using a Hauschild rotary mixer Speedmixer. The sample appeared homogeneous with the water soluble dye evenly distributed throughout the sample. There was no apparent separation in the sample after 18 weeks in ambient lab conditions. When a comparable amount of dyed water was added directly to the silicone elastomer paste and mixed in a Hauschild mixer for 30 s, the combination phase separated with a blue water droplet at rest atop the silicone paste. When the dyed water was pre-mixed with a comparable amount of unmodified μPAA-1 then introduced to the silicone elastomer paste, the aqueous phase (as indicated by the water-soluble dye) failed to disperse evenly into the silicone paste. Likewise, when the pre-mixture of dyed water, unmodified μPAA-1 and Sorbitan monooleate in the same proportions as in Example 6 was introduced to a comparable level of silicone paste, the aqueous phase failed to disperse uniformly in the silicone paste.

This example demonstrates the compatibility of the silicone surface modified hydrogel pastes of this invention with a conventional silicone elastomer paste and therefore introduces a stable means to introduce water and water-soluble actives to a composition having the benefits of a silicone elastomer paste.

### Example 8

A sample was made using the method of Example 2 but having the final composition of: 60.8% Hexamethyldisiloxane, 3.0% μPAA-1, 1.5% Sorbitan monooleate, 30.2% deionized water (with a water soluble dye), and 4.4% A-PDMS.

The filtered product was dried in the vacuum oven overnight at 35° C. and <5 mm Hg. The final dried product dispersed in water but did not form a gel. Upon neutralizing the mixture to a pH of ˜4 with 0.1M KOH, the product formed gel. This demonstrates that the siloxane-modified hydrogels can demonstrate pH responsive swelling behavior.

### Example 9

A sample was made using the method of Example 2 but substituting a 1% aqueous solution of niacinnamide for water and μPAA-cpfor μPAA-1. The final composition of the sample is: 70.9% Hexamethyldisiloxane, 3.7% μPAA-cp, 2.1% Sorbitan monooleate, 17.5% 1% niacinamide in water solution and 5.8% Dimethyl, methyl (aminoethylaminoisobutyl) siloxane.

The filtered product was dried in the vacuum oven overnight at 35° C. and 0.2 in Hg. The final dried product dispersed in water but did not gel. When the neutralized to a pH of ˜4 with 0.1M KOH, the product formed a homogeneous silicone-modified gel. This demonstrates that siloxane-modified hydrogels of this invention can demonstrate pH responsive swelling behavior.

### Example 10

The following procedure was used to produce a dispersion of a siloxane modified hydrogel further coated with a silicone encapsulation layer. The procedure was done in ambient lab conditions.
1. The following ingredients were combined into an 8 oz. straight sided jar: 3.9 parts μPAA-cp, 1.1 part Sorbitan monooleate, and 79.0 parts hexamethyldisiloxane. The mixture was stirred magnetically to achieve homogeneity.
2. 4.1 parts water was added drop wise with mixing. Stirring was continued for a minimum of 10 minutes.
3. 2.9 parts A-PDMS were added drop wise and allowed to mix for an additional 10 minutes.
4. With mixing 8.3 parts methacryloxypropyldimethylsiloxy terminated polydimethylsiloxane (Mn 8,000) were added to the solution in 3 and allowed to mix for 15 minutes.
5. With mixing add 0.6 parts of an initiator complex comprising of tri-n-butyl borane and 1.3 equivalents of methoxypropyl amine.

The filtered product was dried in the vacuum oven overnight at 35° C. and <5 mm Hg. The final dried product was found to disperse in water but did not gel. When neutralized to a pH of ˜4 with 0.1M KOH, the product formed a homogeneous silicone modified gel.

The present invention should not be considered limited to the specific examples described herein, but rather should be understood to cover all embodiments of the invention. Various modifications and equivalent processes, as well as numerous structures and devices, to which the present invention may be applicable will be readily apparent to those of skill in the art. Those skilled in the art will understand that various changes may be made without departing from the scope of the invention, which is not to be considered limited to what is described in the specification.

1. A method for the preparation of siloxane surface-modified hydrogel, comprising:
treating a hydrogel with Component (A), at least one amino-functional organosilicon compound, to form at least one siloxane-coated surface on the hydrogel;
wherein the hydrogel comprises (i) Component (B), at least one organic polymer comprising amine-reactive groups selected from carboxy-functional groups, sulfonic acid-functional groups, epoxy groups, or combinations thereof, the polymer being compatible with water, water-compatible alcohols, or combinations thereof and (ii) Component (C), at least one absorbable solvent selected from water, water-compatible alcohols, and combinations thereof.

2. The method according to claim 1, wherein treatment occurs in the presence of at least one suitable solvent for Component (A), the solvent being selected from hexamethyldisiloxane, octamethyltrisiloxane, decamethyltetrasiloxane, trimethylsilyl-terminated polydimethylsiloxanes having a viscosity of less than 1000 cP at 25° C., capryllylmethyl trisiloxane, octamethylcyclotetrasiloxane, decamethylcyclopentasiloxane, cyclosiloxanes, pentane, hexane, heptane, octane, cyclohexane, toluene, xylenes, ethyl acetate, isododecane, isohexadecane, isodecylneopentanoate, isononyl isononanoate, isoparaffin, isoalkane, 1-ethenyl-3-ethyl-imidazolium hexafluorophosphate, tetrapropyl-ammonium tetracyanoborate, supercritical carbon dioxide, and any combination thereof, and/or wherein Component (A) is selected from poly[dimethyl, methyl (aminoethylaminoisobutyl)] siloxane, poly[dimethyl, methyl (aminoethylaminopropyl)] siloxane, poly[(dimethyl, methylaminopropyl)] siloxane, aminopropyl-terminated polydimethylsiloxane, amino ethylaminopropyl-terminated polydimethylsiloxane, and aminoethylaminoisobutyl-terminated polydimethylsiloxane, and any combination thereof.

3. (canceled)

4. The method according to claim 1, wherein Component (B) has at least 5 mol % of amine-reactive groups or alternatively, at least 10 mol % of amine-reactive groups, and/or wherein Component (B) is selected from carboxy-functional organic polymers, anhydride-functional organic polymers, epoxy-functional organic polymers, polyacrylic acid, poly(meth)acrylic acid; polyacrylic acid and partially crosslinked polyacrylic acid homopolymers, ionomers, and copolymers, and any combination thereof.

5. (canceled)

6. (canceled)

7. The method according to claim 1, wherein Component (C) is selected from water, methanol, ethanol, isopropyl alcohol, ethylene glycol, polyethylene glycol, and any combination thereof.

8. The method according to claim 1, wherein the hydrogel comprises or is treated with Component (D), at least one active ingredient selected from personal care, healthcare, or agricultural active ingredients, wherein Component (D) is encapsulated within the siloxane-coated surface.

9. The method according to claim 8, wherein Component (D) is at least one active ingredient selected from Vitamin C, green tea extract, lidocaine, nicotine, niacinnamide, salicylic acid, ketoprofen, ketoconazole, urea, ammonium nitrate, potassium nitrate, sodium nitrate, potassium phosphate, and ammonium phosphate, and any combination thereof.

10. The method according to claim 1, wherein the hydrogel is treated in the presence of Component (E), at least one surfactant selected from Tergitol 15-s-3, Tergitol 15-s-40, sorbitan monooleate polyglycol-modified trimethsilylated silicate, polyglycol-modified siloxanes, polyglycol-modified silicas, ethoxylated quaternary ammonium salt solutions, cetyltrimethylammonium chloride solutions, and any combination thereof.

11. The method according to claim 1, comprising treating the siloxane-coated hydrogel with Component (F), at least one free-radical polymerizable compound and Component (G), at least one organoborane free radical initiator; wherein such treatment occurs in the presence of oxygen.

12. The method according to claim 11, wherein Component (F) is selected from methacrylate-functional polydimethylsiloxanes and resins, acrylate-functional polydimethylsiloxanes and resins, and any combination thereof and/or wherein Component (G) is selected from triethylborane-propanediamine, triethylborane-butylimidazole, triethylborane-methoxypropylamine complexes, tri-n-butyl methoxypropyl amine complexes, and any combination thereof.

13. (canceled)

14. A siloxane surface-modified hydrogel prepared according to the method of claim 1.

15. A paste prepared from a hydrogel of claim 14.

16. A method for the preparation of siloxane surface-modified hydrogel microparticles, comprising:
treating hydrogel microparticles with Component (A), at least one amino-functional organosilicon compound, to form microparticles having at least one siloxane-coated surface;
wherein the microparticles comprise Component (B), at least one organic polymer comprising amine-reactive groups selected from carboxy-functional groups, sulfonic acid-functional groups, epoxy groups, or combinations thereof, the polymer being compatible with water, water-compatible alcohols, or combinations thereof; and
wherein treatment occurs in the presence of Component (C), at least one absorbable solvent selected from water, water-compatible alcohols, and combinations thereof.

17. The method according to claim 16, wherein treatment occurs in the presence of at least one suitable solvent for Component (A), the solvent being selected from hexamethyldisiloxane, octamethyltrisiloxane, decamethyltetrasiloxane, trimethylsilyl-terminated polydimethylsiloxanes having a viscosity of less than 1000 cP at 25° C., capryllylmethyl trisiloxane, octamethylcyclotetrasiloxane, decamethylcyclopentasiloxane, cyclosiloxanes, pentane, hexane, heptane, octane, cyclohexane, toluene, xylenes, ethyl acetate, isododecane, isohexadecane, isodecylneopentanoate, isononyl isononanoate, isoparaffin, isoalkane, 1-ethenyl-3-ethyl-imidazolium hexafluorophosphate, tetrapropyl-ammonium tetracyanoborate, supercritical carbon dioxide, and any combination thereof, and/or wherein Component(A) is selected from poly[dimethyl, methyl (aminoethylaminoisobutyl)] siloxane, poly[dimethyl, methyl (aminoethylaminopropyl)] siloxane, poly[(dimethyl, methylaminopropyl)] siloxane, aminopropyl-terminated polydimethylsiloxane, amino ethylaminopropyl-terminated polydimethylsiloxane, and aminoethylaminoisobutyl-terminated polydimethylsiloxane, and any combination mixtures thereof.

18. (canceled)

19. The method according to claim 16, wherein Component (B) has at least 5 mol % of amine-reactive groups or alternatively, at least 10 mol % of amine-reactive groups and/or wherein Component (B) is selected from carboxy-functional organic polymers, anhydride-functional organic polymers, epoxy-functional organic polymers, polyacrylic acid, poly(meth)acrylic acid; polyacrylic acid and partially crosslinked polyacrylic acid homopolymers, ionomers, and copolymers, and any combination thereof and/or wherein Component (C) is selected from water, methanol, ethanol, isopropyl alcohol, ethylene glycol, polyethylene glycol, and any combination thereof.

20. (canceled)

21. (canceled)

22. (canceled)

23. The method according to claim 16, wherein the hydrogel microparticles comprise or are treated with Component (D), at least one active ingredient selected from personal care, healthcare, or agricultural active ingredients, wherein Component (D) is encapsulated within the siloxane-coated surface.

24. The method according to claim 23, wherein Component (D) is at least one active ingredient selected from Vitamin C, green tea extract, lidocaine, nicotine, niacinnamide, salicylic acid, ketoprofen, ketoconazole, urea, ammonium nitrate, potassium nitrate, sodium nitrate, potassium phosphate, and ammonium phosphate, and any combination thereof.

25. The method according to claim 16, wherein the hydrogel microparticles are treated in the presence of Component (E), at least one surfactant selected from Tergitol 15-s-3, Tergitol 15-s-40, sorbitan monooleate, polyglycol-modified trimethsilylated silicate, polyglycol-modified siloxanes, polyglycol-modified silicas, ethoxylated quaternary ammonium salt solutions, cetyltrimethylammonium chloride solutions, and any combination thereof.

26. The method according to claim 16, comprising treating the siloxane-coated hydrogel microparticles with Component (F), at least one free-radical polymerizable compound selected from methacrylate-functional polydimethylsiloxanes and resins, acrylate-functional polydimethylsiloxanes and resins, and any combination thereof, and Component (G), at least one organoborane free radical initiator selected from triethylborane-propanediamine, triethylborane-butylimidazole, triethylborane-methoxypropylamine complexes, and tri-n-butyl methoxypropyl amine complexes, and any combination thereof; wherein such treatment occurs in the presence of oxygen.

27. (canceled)

28. (canceled)

29. Hydrogel microparticles prepared according to the method of claim 15.

30. A paste prepared from hydrogel microparticles of claim 29.
